(19) United States
(12) Patent Application Publication
(10) Pub. No.: US 2012/0214188 A1
(43) Pub. Date: Aug. 23, 2012

(54) LIGHT-ACTIVATED ION CHANNEL MOLECULES AND USES THEREOF

(76) Inventors: Nathan Klapoetke, Cambridge, MA (US); Amy Chuong, Cambridge, MA (US); Brian Yichiun Chow, Cambridge, MA (US); Edward Boyden, Chestnut Hill, MA (US); Xue Han, Chestnut Hill, MA (US); Gane Ka-Shu Wong, Edmonton (CA)

(21) Appl. No.: 13/295,479

(22) Filed: Nov. 14, 2011

Related U.S. Application Data
(60) Provisional application No. 61/413,161, filed on Nov. 12, 2010.

(51) Int. Cl. C12N 13/00; C12Q 1/02; C12N 15/12; C12N 15/63; C07K 14/435; C12N 5/07

(52) U.S. Cl. 435/29; 530/350; 435/325; 536/23.5; 435/320.1; 435/173.1

(57) ABSTRACT

The invention, in some aspects relates to compositions and methods for altering cell activity and function and the use of light-activated ion channels (LAICs).

### RELATED APPLICATIONS

This application claims the benefit under 35 U.S.C. §119(e) of U.S. Provisional application Ser. No. 61/413,161 filed Nov. 12, 2010, the disclosure of which is incorporated by reference herein in its entirety.

### GOVERNMENT INTEREST

This invention was made with government support under grants 1DP2OD002002, and 1R01DA029639 awarded by the National Institutes of Health, and grant EFRI 0835878 and Career Award CBET 1053233 awarded by the National Science Foundation. The United States government has certain rights in the invention.

### FIELD OF THE INVENTION

The invention, in some aspects relates to compositions and methods for altering cell activity and function and the use of light-activated ion channels.

### BACKGROUND OF THE INVENTION

Altering and controlling cell membrane and subcellular region ion permeability has permitted examination of characteristics of cells, tissues, and organisms. Light-driven pumps and channels have been used to silence or enhance cell activity and their use has been proposed for drug screening, therapeutic applications, and for exploring cellular and subcellular function.

Molecular-genetic methods for preparing cells that can be activated (e.g., depolarized) or inactivated (e.g., hyperpolarized) by specific wavelengths of light have been developed (see, for example, Han, X. and E. S. Boyden, 2007, PLoS ONE 2, e299). It has been identified that the light-activated cation channel channelrhodopsin-2 (ChR2), and the light-activated chloride pump halorhodopsin (Halo/NpHR), when transgenically expressed in cell such as neurons, make them sensitive to being activated by blue light, and silenced by yellow light, respectively (Han, X. and E. S. Boyden, 2007, PLoS ONE 2(3): e299; Boyden, E. S., et. al., 2005, Nat Neurosci. 2005 September; 8(9):1263-8. Epub 2005 Aug. 14). Previously identified light-activated pumps and channels have been restricted to activation by particular wavelengths of light, thus limiting their usefulness.

### SUMMARY OF THE INVENTION

The invention, in part, relates to isolated light-activated ion channel (LAIC) polypeptides and methods for their preparation and use. The invention also includes isolated nucleic acid sequences that encode light-driven ion channels of the invention as well as vectors and constructs that comprise such nucleic acid sequences. In addition, the invention in some aspects includes expression of light-activated ion channel polypeptides in cells, tissues, and organisms as well as methods for using the light-activated ion channels to alter cell and tissue function and for use in diagnosis and treatment of disorders.

The invention, in part, also relates to methods for adjusting the voltage potential of cells, subcellular regions, or extracellular regions. Some aspects of the invention include methods of incorporating at least one nucleic acid sequence encoding a light-driven ion channel into at least one target cell, subcellular region, or extracellular region, the ion channel functioning to change transmembrane passage of ions in response to a specific wavelength of light. Exposing an excitable cell that includes an expressed light-driven ion channel of the invention to a wavelength of light that activates the channel, may result in depolarization of the excitable cell. By contacting a cell that includes a LAIC of the invention with particular wavelengths of light, the cell is depolarized. A plurality of light-activated ion channels activated by different wavelengths of light may be used to achieve multi-color depolarization.

In some embodiments, the invention comprises a method for the expression of certain classes of genes encoding for light-driven ion channels, in genetically-targeted cells, to allow millisecond-timescale generation of depolarizing current in response to pulses of light. These channels can be genetically-expressed in specific cells (e.g., using a virus) and then used to control cells in intact organisms (including humans) as well as cells in vitro, in response to pulses of light. Given that these channels have different activation spectra from one another and from the state of the art (e.g., ChR2/VChR1), they also allow multiple colors of light to be used to depolarize different sets of cells in the same tissue, simply by expressing channels with different activation spectra genetically in different cells, and then illuminating the tissue with different colors of light.

In some aspects, the invention uses eukaryotic channelrhodpsins, such as Tetraselmis striata, Tetraselmis chuii, and Brachiomonas submarina rhodopsin to depolarize excitable cells. These channelrhodpsins may also be used to modify the pH of cells, or to introduce cations as chemical transmitters.

The ability to optically perturb, modify, or control cellular function offers many advantages over physical manipulation mechanisms, such as speed, non-invasiveness, and the ability to easily span vast spatial scales from the nanoscale to macroscale. One such approach is an opto-genetic approach, in which heterologously expressed light-activated membrane polypeptides such as a LAIC of the invention, are used to move ions with various spectra of light.

According to one aspect of the invention, isolated ultraviolet-light-activated ion channel polypeptides are provided, wherein the ion channel when expressed in a membrane is activated by contact with ultraviolet light. In certain embodiments, the ultraviolet light has a wavelength of about 340 nm to about 400 nm.

According to another aspect of the invention, a cell that includes any embodiment of the aforementioned isolated ultraviolet-light-activated ion channel polypeptides. In some embodiments, the cell is an excitable cell. In some embodiments, the cell is a mammalian cell. In some embodiments, the cell is in vitro, ex vivo, or in vivo. In certain embodiments, the cell also includes one, two, three, four, or more additional light-activated ion channels, wherein at least one, two, three, four, or more of the additional light-activated ion channels is activated by contact with light that is not ultraviolet light and is not activated by light having a wavelength between about 340 and 400 nm.

According to another aspect of the invention, nucleic acid sequences that encode any embodiment of the aforementioned isolated ultraviolet-light-activated ion channel polypeptide are provided. In some embodiments, the sequence comprises the sequence set forth as SEQ ID NO:1, SEQ ID NO:3, or SEQ ID NO:5. In some embodiments, the nucleic acid sequence is a mammalian codon-optimized DNA sequence.

According to another aspect of the invention, a vector comprising the any embodiment of an aforementioned nucleic acid sequence is provided. In certain embodiments, the nucleic acid sequence is operatively linked to a promoter sequence. In some embodiments, the vector also includes one, two, or more nucleic acid signal sequences operatively linked to the nucleic acid sequence encoding the ultraviolet-light-activated ion channel. In some embodiments, the vector is a plasmid vector, cosmid vector, viral vector, or an artificial chromosome.

According to yet another aspect of the invention, a cell that includes any embodiment of the aforementioned vectors is provided. In certain embodiments of the aforementioned aspects of the invention, the ultraviolet-light-activated ion channel polypeptide sequence includes conserved amino acids that correspond to G129, 5133, T136, G141, and 5169 of the amino acid sequence of ChR66, set forth herein as (SEQ ID NO:2). In some embodiments, the ultraviolet-light-activated ion channel comprises the amino acid sequence of a wild-type or modified phytoplankton rhodopsin. In some embodiments, the phytoplankton is a member of the genus Tetraselmis. In some embodiments, the sequence of the light-activated ion channel polypeptide comprises an amino acid sequence set forth as SEQ ID NO:2, SEQ ID NO:4, or SEQ ID NO:6. In certain embodiments, the ultraviolet-light-activated channel is activated and the cell depolarized when the channel is contacted with ultraviolet-light under suitable conditions for depolarization. In some embodiments, contacting the channel with ultraviolet light under suitable conditions modifies the pH of the interior of the cell. In some embodiments, cations enter the cell when the channel is contacted with ultraviolet light under suitable conditions. In certain embodiments, any of aforementioned cell is a normal cell. In some embodiments, the cell is a mammalian cell.

According to another aspect of the invention, methods of depolarizing a cell are provided. The methods include contacting a cell comprising an isolated ultraviolet-light-activated ion channel with an ultraviolet light under conditions suitable to depolarize the cell and depolarizing the cell. In some embodiments, the ultraviolet-light-activated ion channel activates in response to ultraviolet light in the range of about 340 nm to about 400 nm. In certain embodiments, the ultraviolet-light-activated ion channel polypeptide comprises conserved amino acids that correspond to G129, 5133, T136, G141, and 5169 of the amino acid sequence of ChR66, set forth herein as (SEQ ID NO:2). In some embodiments, the ultraviolet-light-activated ion channel polypeptide sequence comprises an amino acid sequence of a wild-type or modified phytoplankton rhodopsin. In some embodiments, the phytoplankton is a member of the genus Tetraselmis. In some embodiments, the ultraviolet-light-activated ion channel polypeptide is encoded by the nucleic acid sequence set forth as SEQ ID NO:1, SEQ ID NO:3, or SEQ ID NO:5. In certain embodiments, the amino acid sequence of the ultraviolet-light-activated ion channel polypeptide sequence includes the sequence set forth as SEQ ID NO:2, SEQ ID NO:4, or SEQ ID NO:6. In some embodiments, the ultraviolet-light-activated ion channel is not significantly activated in response to contact with non-ultraviolet light. In some embodiments, the cell is a nervous system cell, a cardiac cell, a circulatory system cell, a visual system cell, an auditory system cell, or a muscle cell. In some embodiments, the cell is a mammalian cell. In certain embodiments, the cell also includes one, two, three, or more additional light-activated ion channel polypeptides, wherein at least one, two, three, four, or more of the additional light-activated ion channel polypeptides is activated by contact with light having a non-ultraviolet light wavelength and is not significantly activated by light having a wavelength between about 340 to 400 nm. In some embodiments, the cell is in a subject and depolarizing the cell diagnoses or assists in a diagnosis of a disorder in the subject. In some embodiments, the cell is in a subject and depolarizing the cell treats a disorder in the subject.

According to another aspect of the invention, methods of assessing the effect of a candidate compound on a cell are provided. The methods include contacting a test cell that includes an isolated ultraviolet-light-activated ion channel with ultraviolet light under conditions suitable for depolarization of the cell; contacting the test cell with a candidate compound; and c) identifying the presence or absence of a change in depolarization or a change in a depolarization-mediated cell characteristic in the test cell contacted with the ultraviolet light and the candidate compound compared to depolarization or a depolarization-mediated cell characteristic, respectively, in a control cell contacted with the ultraviolet light and not contacted with the candidate compound; wherein a change in depolarization or a depolarization-mediated cell characteristic in the test cell compared to the control indicates an effect of the candidate compound on the test cell. In some embodiments, the ultraviolet light is in the range of about 340 nm to about 400 nm. In certain embodiments, the effect of the candidate compound is an effect on the depolarization of the test cell. In some embodiments, the effect of the candidate compound is an effect on a depolarization-mediated cell characteristic in the test cell. In some embodiments, the method also includes characterizing the change identified in the depolarization or the depolarization-mediated cell characteristic. In certain embodiments, the ultraviolet-light-activated ion channel polypeptide sequence comprises conserved amino acids that correspond to G129, 5133, T136, G141, and 5169 of the amino acid sequence of ChR66, set forth herein as (SEQ ID NO:2). In some embodiments, the ultraviolet-light-activated ion channel polypeptide sequence includes an amino acid sequence of a wild-type or modified phytoplankton rhodopsin. In some embodiments, the phytoplankton is a member of the genus Tetraselmis. In some embodiments, the ultraviolet-light-activated ion channel is encoded by the nucleic acid sequence set forth as SEQ ID NO:1, SEQ ID NO:3, or SEQ ID NO:5. In certain embodiments, the amino acid sequence of the ultraviolet-light-activated ion channel polypeptide includes an amino acid sequence forth as SEQ ID NO:2, SEQ ID NO:4, or SEQ ID NO:6. In some embodiments, the ultraviolet-light-activated ion channel does not activate in response to contact with non-ultraviolet light. In some embodiments, a depolarization-mediated cell characteristic is an action potential. In some embodiments, a depolarization-mediated cell characteristic release of a neurotransmitter. In certain embodiments, the cell is a nervous system cell, a cardiac cell, a circulatory system cell, a visual system cell, an auditory system cell, a muscle cell, or another excitable cell. In some embodiments, the cell is a mammalian cell. In some embodiments, the cell also includes one, two, three, or more additional light-activated ion channel polypeptides, wherein at least one, two, three, four, or more of the additional light-activated ion channel polypeptides is activated by contact with light having a non-ultraviolet light wavelength and is not activated by contact with ultraviolet light having a wavelength between 340 nm and 400 nm. According to yet another aspect of the invention, methods of treating a disorder in a subject are provided. The methods include administering to a subject in need of such treatment, a therapeutically effective amount of an ultraviolet-light-activated ion channel, to treat the disorder and contacting the cell with ultraviolet light and activating the ultraviolet-light-activated ion channel in the cell under conditions sufficient to depolarize the cell, wherein depolarizing the cell treats the disorder in the subject. In certain embodiments, the ultraviolet-light-activated ion channel is administered in the form of a cell, wherein the cell expresses the ultra-violet-light-activated ion channel, or in the form of a vector, wherein the vector comprises a nucleic acid sequence encoding the ultraviolet-light-activated ion channel and the administration of the vector results in expression of the ultraviolet-light-activated ion channel in a cell in the subject. In some embodiments, the vector further comprises a signal sequence. In some embodiments, the vector further comprises a cell-specific promoter. In certain embodiments, the disorder is a neurological disorder, a visual system disorder, a circulatory system disorder, a musculoskeletal system disorder, or an auditory system disorder. In some embodiments, the method also includes administering an additional therapeutic composition to the subject. In some embodiments, depolarizing the cell modulates a depolarization-mediated cell characteristic. In certain embodiments, a depolarization-mediated cell characteristic is an action potential. In some embodiments, a depolarization-mediated cell characteristic release of a neurotransmitter. In some embodiments, the ultraviolet-light-activated ion channel activates in response to ultraviolet light in the range of about 340 nm to about 400 nm. In certain embodiments, the ultraviolet-light-activated ion channel polypeptide sequence comprises conserved amino acids that correspond to G129, 5133, T136, G141, and 5169 of the amino acid sequence of ChR66, set forth herein as (SEQ ID NO:2). In some embodiments, the ultraviolet-light-activated ion channel polypeptide sequence comprises an amino acid sequence of a wild-type or modified phytoplankton rhodopsin. In some embodiments, the phytoplankton is a member of the genus Tetraselmis. In some embodiments, the ultraviolet-light-activated ion channel is encoded by a nucleic acid sequence set forth as SEQ ID NO:1, SEQ ID NO:3, or SEQ ID NO:5. In certain embodiments, the amino acid sequence of the ultraviolet-light-activated ion channel is set forth as SEQ ID NO:2, SEQ ID NO:4, or SEQ ID NO:6. In some embodiments, the ultraviolet-light-activated ion channel does not activate in response to contact with light that is not ultraviolet light. In some embodiments, the cell is a nervous system cell, a neuron, a cardiac cell, a circulatory system cell, a visual system cell, an auditory system cell, or a muscle cell. In certain embodiments, the cell is a mammalian cell. In some embodiments, the cell also includes one, two, three, or more additional light-activated ion channel polypeptides, wherein at least one, two, three, four, or more of the additional light-activated ion channel polypeptides is activated by contact with light having a non-ultraviolet light wavelength and is not activated by contact with ultraviolet light having a wavelength between 340 nm and 400 nm.

According to another aspect of the invention, isolated light-activated ion channel polypeptides are provided, wherein when present in a cell or membrane, the isolated light-activated ion channel is activated by contact light having a wavelength between about 385 nm to about 530 nm, and wherein the light-activated ion channel has effectively zero Ca⁺⁺ conductance when activated by light. In some embodiments, the wavelength of light that activates the light-activated ion channel is about 385 nm to about 530 nm, between about 400 nm to about 500 nm, between about 450 nm to about 500 nm, or between 465 nm to 475 nm. In some embodiments, the light-activated ion channel polypeptide sequence comprises a wild-type or modified algal light-activated ion channel polypeptide sequence. In certain embodiments, the algae is a member of the genus Brachiomonas. In some embodiments, the light-activated ion channel polypeptide sequence includes the sequence set forth as SEQ ID NO:8.

According to another aspect of the invention, a cell that includes any embodiment of an aforementioned isolated light-activated ion channel polypeptide is provided. In some embodiments, the cell is an excitable cell. In some embodiments, the cell is a mammalian cell. In certain embodiments, the cell is in vitro, ex vivo, or in vivo. In some embodiments, the cell also includes one, two, three, four, or more additional heterologous light-activated ion channels, wherein at least one, two, three, four, or more of the additional light-activated ion channels is activated by contact with light that is not ultraviolet light.

According to another aspect of the invention, a nucleic acid sequence that encodes any embodiment of an aforementioned isolated light-activated ion channel polypeptide is provided. In some embodiments, the nucleic acid sequence is a mammalian codon-optimized DNA sequence. In certain embodiments, nucleic acid sequence is set forth as SEQ ID NO:7.

According to another aspect of the invention, a vector that includes any embodiment of an aforementioned nucleic acid sequence is provided. In some embodiments, the nucleic acid sequence is operatively linked to a promoter sequence. In some embodiments, the vector also includes one, two, or more nucleic acid signal sequences operatively linked to the nucleic acid sequence encoding the light-activated ion channel. In some embodiments, the vector is a plasmid vector, cosmid vector, viral vector, or an artificial chromosome.

According to another aspect of the invention, a cell that includes any embodiment of an aforementioned vector is provided. In certain embodiments, the cell also includes one, two, three, four, or more additional heterologous light-activated ion channels, wherein at least one, two, three, four, or more of the additional light-activated ion channels is activated by contact with light having a wavelength not in the range of about 385 nm to about 530 nm. In some embodiments of any of the aforementioned aspects of the invention, the light-activated channel is activated and the cell depolarized when the channel is contacted with the light under suitable conditions for depolarization. In some embodiments, contacting the channel with light under suitable conditions modifies the pH of the interior of the cell. In some embodiments, cations enter the cell when the channel is contacted with ultraviolet light under suitable conditions. In certain embodiments, the cell is a mammalian cell.

According to yet another aspect of the invention, methods of depolarizing a cell are provided. The methods include expressing in a cell a light-activated ion channel that activates in response to contact with a light having a wavelength between about 385 nm to about 530 nm, and has effectively zero Ca⁺⁺ conductance when activated by light; and contacting the isolated light-activated ion channel with a light that activates the light-activated channel and depolarizing the cell. In some embodiments, the light-activated ion channel has the amino acid sequence of wild-type or modified algal light-activated ion channel. In some embodiments, the algae is a member of the genus Brachiomonas. In certain embodiments, the light-activated ion channel is encoded by the nucleic acid sequence set forth as SEQ ID NO:7. In some embodiments, the amino acid sequence of the light-activated ion channel is set forth as SEQ ID NO:8. In some embodiments, the light-activated ion channel does not significantly activate in response to contact with light outside the range of about 385 nm to about 530 nm. In some embodiments, the cell is a nervous system cell, a cardiac cell, a circulatory system cell, a visual system cell, or an auditory system cell. In certain embodiments, the cell is a mammalian cell. In some embodiments, the cell is a normal cell. In some embodiments, the cell also includes one, two, three, or more additional heterologous light-activated ion channel polypeptides, wherein at least one, two, three, four, or more of the additional light-activated ion channel polypeptides is activated by light but is not activated by contact with light in the range of about 385 nm to about 530 nm.

According to yet another aspect of the invention, methods of identifying an effect of a candidate compound on a cell are provided. The methods include contacting a test cell comprising an isolated light-activated ion channel that activates in response to contact with a light having a wavelength between about 385 nm and about 530 nm, under conditions suitable for depolarization of the cell; contacting the test cell with a candidate compound; and identifying the presence or absence of a change in depolarization or a change in a depolarization-mediated cell characteristic in the test cell contacted with the light and the candidate compound compared to depolarization or a depolarization-mediated cell characteristic, respectively, in a control cell contacted with a light the light and not contacted with the candidate compound; wherein a change in depolarization or a depolarization-mediated cell characteristic in the test cell compared to the control indicates an effect of the candidate compound on the test cell. In some embodiments, the effect of the candidate compound is an effect on the depolarization of the test cell. In certain embodiments, the effect of the candidate compound is an effect on a depolarization-mediated cell characteristic in the test cell. In some embodiments, the method also includes characterizing the change identified in the depolarization or depolarization-mediated cell characteristic. In some embodiments, the light-activated ion channel polypeptide sequence comprises an amino acid sequence of a wild-type or modified algal light-activated ion channel. In certain embodiments, the algae is a member of the genus Brachiomonas. In some embodiments, the light-activated ion channel is encoded by the nucleic acid sequence set forth as SEQ ID NO:7. In some embodiments, the amino acid sequence of the light-activated ion channel is set forth as SEQ ID NO:8. In some embodiments, the light-activated ion channel does not activate in response to contact with light that does not have a wavelength of about 385 nm to about 530 nm. In some embodiments, the cell is a nervous system cell, a cardiac cell, a circulatory system cell, a visual system cell, an auditory system cell, or a muscle cell. In some embodiments, the cell is a mammalian cell. In some embodiments, the cell is a normal cell. In some embodiments, the cell also includes one, two, three, or more additional heterologous light-activated ion channel polypeptides, wherein at least one, two, three, four, or more of the additional light-activated ion channel polypeptides is activated by contact with light having a wavelength that is not in the range of about 385 nm to about 530 nm. In some embodiments, one, two, three, or more of the additional light-activated ion channel polypeptides is not activated by contact with light having a wavelength that between about 385 nm to about 530 nm, between about 400 nm to about 500 nm, between about 450 nm to about 500 nm, or between 465 nm to 475 nm.

According to yet another aspect of the invention, methods of treating a disorder in a subject are provided. The methods include: administering to a subject in need of such treatment, a therapeutically effective amount of a light-activated ion channel that under suitable conditions is activated by contact light having a wavelength between about 385 nm to about 530 nm, and wherein the light-activated ion channel has about zero Ca⁺⁺ conductance when in a cell and activated by light, to treat the disorder. In some embodiments, the light-activated ion channel is administered in the form of a cell, wherein the cell expresses the light-activated ion channel; or in the form of a vector, wherein the vector comprises a nucleic acid sequence encoding the light-activated ion channel and the administration of the vector results in expression of the light-activated ion channel in a cell in the subject. In some embodiments, the method also includes contacting the cell with light and activating the light-activated ion channel in the cell. In some embodiments, the method also includes administering an additional therapeutic composition to the subject. In some embodiments, depolarizing the cell modulates a depolarization-mediated cell characteristic. In some embodiments, the light-activated ion channel polypeptide sequence includes the amino acid sequence of wild-type or modified algal light-activated ion channel. In some embodiments, the algae is a member of the genus Brachiomonas. In some embodiments, the ion channel is encoded by the nucleic acid sequence set forth as SEQ ID NO:7. In some embodiments, the amino acid sequence of the light-activated ion channel is set forth as SEQ ID NO:8. In some embodiments, the light-activated ion channel does not significantly activate in response to contact with light that does not have a wavelength of about 385 nm to about 530 nm. In some embodiments, the cell is a nervous system cell, a cardiac cell, a circulatory system cell, a visual system cell, an auditory system cell, or a muscle cell. In some embodiments, the cell is a mammalian cell. In some embodiments, the cell is a normal cell. In some embodiments, the cell also includes one, two, three, or more additional heterologous light-activated ion channel polypeptides, wherein at least one, two, three, four, or more of the additional light-activated ion channel polypeptides is activated by contact with light having a wavelength that is not in the range of about 385 nm to about 530 nm. In some embodiments, one, two, three, or more of the additional light-activated ion channel polypeptides is not activated by contact with light having a wavelength that between about 385 nm to about 530 nm, between about 400 nm to about 500 nm, between about 450 nm to about 500 nm, or between 465 nm to 475 nm.

### BRIEF DESCRIPTION OF THE DRAWINGS

FIG. 1 is a graph showing light-activated normalized activity for ChR2 and ChR66, and ChR65 across a range of wavelengths. The action spectrum was taken in HEK293FT cells. Equal photon flux was used at each wavelength. The graph shows a shift toward the UV range for the ChR66 LAIC activation and peak compared to ChR2.

FIG. 2 shows graphs of comparison data of inward photocurrent elicited at peak wavelength sensitivity of channelrhodopsin in cultured hippocampal neuron. 470±11 nm light was used for ChR2; 434±9 nm light was sued for ChR66, ChR68, and ChR77. All illuminations consisted of 500 ms pulse of light at 10 mW mm⁻². KGC and ER2 are Kir2.1 trafficking sequences used to improve membrane expression of channelrhodopsin. FIG. 2A shows results with Iₘₐₓ defined as the maximum photocurrent during the 500 nm pulse interval. FIG. 2B shows results with Iₘᵢₙ defined as the minimum photocurrent at the end of the 500 ms pulse.

FIG. 3 shows graphs of comparisons of inward photocurrent at 406±7 nm 10 mW mm⁻² in cultured hippocampal neuron. KGC-ER2 trafficking sequences was sued for all constructs. FIG. 3A shows peak photocurrent response to 500 ms pulse for ChR2, ChR66, and ChR68. FIG. 3B shows minimum photocurrent at the end of 500s for ChR2, ChR66, and ChR68. FIG. 3C shows a comparison of results for ChR2 and ChR66 of 406 nm photon dose modulation with fixed power at 10 mW mm⁻² with varying pulse duration.

FIG. 4 shows a recording trace and a graph illustrating photocurrent kinetics measured at 406±7 nm 10 mW mm⁻² in cultured hippocampal neuron. A KGC-ER2 trafficking sequence was used for all constructs. FIG. 4A shows representative photocurrent trace of ChR2 and ChR66 in voltage clamp mode. FIG. 4B is a graph showing comparison of turn on kinetics measured as single exponential fit of 10-90% initial transient photocurrent as measured for ChR2, ChR66, and ChR68.

FIG. 5 is a trace diagram showing example traces of high fidelity spike train elicited in cultured hippocampal neuron at 406 nm with ChR66. ChR66 has fast channel closing kinetics around 6.5 ms.

FIG. 6 is a trace diagram showing example traces of driving spike train with ChR66 using ultraviolet light at 365 nm in cultured hippocampal neuron.

FIG. 7 is a graph showing Fura2 calcium imaging of ChR2 and ChR65 in HEK293. Control is untransfected cells. Extracellular medium is modified tyrode with 90 mM calcium. ChR65 does not exhibit any calcium permeability after 10 seconds of blue light (470 nm) illumination.

FIG. 8 is a photomicrographic image showing ChR66 expression in a cultured hippocampal neuron. CaMKII-ChR66-KGC-YPF-ER2 was transfected using calcium phosphate.

FIG. 9 is a photomicrographic image showing ChR66 and ChR77 expression in visual cortex. KGC-ER2 trafficking sequence was used for both constructs. FIGS. 9A-C show images of ChR66 expressed in visual cortex. FIG. 9A shows an image of the mouse visual cortex showing localization of the expressed ChR66 neurons via GFP fluorescence. FIGS. 9B and C show higher resolution images of the ChR66 expressed in individual cells of the visual cortex. FIG. 9D shows an image of the mouse visual cortex showing localization of the expressed ChR77 cells seen via GFP fluorescence. FIGS. 9E and F show a higher resolution image of the ChR77 expression in individual cells of the visual cortex.

### BRIEF DESCRIPTION OF THE SEQUENCES

SEQ ID NO:1 is the mammalian codon-optimized DNA sequence that encodes the wild-type Tetraselmis striata channelrhodopsin, also referred to herein as ChR66:

atgttcgccatcaaccccgaatatatgaatgagactgtgctgctggacgaatgcaccccaatctacctggatattggaccactgtgg
gagcaggtggtggctagggtgactcagtggttcggagtgatcctgtctctggtgtttctgatctactacatctggaacacttacaagg
ctacctgtgggtgggaggaactgtacgtgtgcacagtggagttctgtaagatcatcatcgaactgtacttcgagtatactccccctg
ccatgatcttccagacaaatggacaagtgactccctggctgcggtatgctgagtggctgctgacatgccctgtgatcctgattcacc
tgtctaacattactgggctgaatgacgattacagtggccgcaccatgagcctgatcacatccgacctgggcggaatttgtatggctg
tgaccgccgctctgagcaagggatggctgaaagccctgttctttgtgatcgggtgcggctacggagccagcaccttctacaacgc
cgcttgtatctacattgagtcctactataccatgccacagggaatttgcaggagactggtgctgtggatggccggggtgttctttaca
tcctggttcatgtttcctggcctgttcctggctggaccagaaggaacccaggccctgtcttgggctggaaccacaatcggacatac
agtggccgacctgctgagtaagaacgcttggggaatgatcgggcactttctgagggtggagatccacaagcatatcatcatccat
ggagatgtgcggcgccccgtgacagtgaaggctctggggaggcaggtgagcgtgaattgtttcgtggacaaagaggaagagg
aagaggatgagagaatc.

SEQ ID NO: 2 is the amino acid sequence of the wild-type Tetraselmis striata channelrhodopsin, also referred to herein as ChR66:

MFAINPEYMNETVLLDECTPIYLDIGPLWEQVVARVTQWFGVILSLVFL
IYYIWNTYKATCGWEELYVCTVEFCKIIIELYFEYTPPAMIFQTNGQVT
PWLRYAEWLLTCPVILIHLSNITGLNDDYSGRTMSLITSDLGGICMAVT
AALSKGWLKALFFVIGCGYGASTFYNAACIYIESYYTMPQGICRRLVLW
MAGVFFTSWFMFPGLFLAGPEGTQALSWAGTTIGHTVADLLSKNAWGMI
GHFLRVEIHKHIIIHGDVRRPVTVKALGRQVSVNCFVDKEEEEEDERI.

SEQ ID NO:3 is the mammalian codon-optimized DNA sequence that encodes wild-type Tetraselmis chuii channelrhodopsin, also referred to herein as ChR68:

atgggcttccagctgaaccccgaatatctgaatgagacaattctgctggacgattgcactccaatctacctgaacgtgggaccact
gtgggagcagaaggtggctcggggaactcagtggttcggcgtgatcctgagcctggccttcctgatctactatatttggatcacat
ataaggctaccacatgtgggtgggaggaactgtacgtgtgcactattgagttctgtaaaattgtgatcgaactgtatttcgagtttt
ccccccctgccatgatctaccagaccaatggagaagtgacaccctggctgagatatgctgagtggctgctgacctgccctgtgatt
ctgatccacctgtctaacatcacaggcctgaatgacgattacagtggaagaactatgtctctgattaccagtgacctgggcggaatc
tgtatggctgtgaccagcgccctgtccaagggatggctgaaatggctgttctttgtgatcggctgctgttacggagccagcactttc
tatcacgccgctctgatctacatcgaatcctactataccatgccacatggagtgtgcaagaacatggtgctggccatggccgccgt
gttcttcacctcttggttcatgtttcctgggctgtttctggctgggccagagggcacaaacgccctgagctgggctgggtccacaa
tcggccatactgtggccgatctgctgagtaagaatgcttggggaatgattgggcacttcctgagactggaaatccacaaacatatca
ttatccatggggacgtgaggagacccattactgtgaataccctgggccgggaggtgaccgtgtatgttttgtggataaagaggaa
gaggacgaagatgagcgcatc.

SEQ ID NO:4 is the amino acid sequence of wild-type Tetraselmis chuii channelrhodopsin, also referred to herein as ChR68:

MGFQLNPEYLNETILLDDCTPIYLNVGPLWEQKVARGTQWFGVILSLAF
LIYYIWITYKATTCGWEELYVCTIEFCKIVIELYFEFSPPAMIYQTNGE
VTPWLRYAEWLLTCPVILIHLSNITGLNDDYSGRTMSLITSDLGGICMA
VTSALSKGWLKWLFFVIGCCYGASTFYHAALIYIESYYTMPHGVCKNMV
LAMAAVFFTSWFMFPGLFLAGPEGTNALSWAGSTIGHTVADLLSKNAWG
MIGHFLRLEIHKHIIIHGDVRRPITVNTLGREVTVSCFVDKEEEDEDER
I.

SEQ ID NO:5 is the mammalian codon-optimized DNA sequence that encodes wild-type Tetraselmis cordiformis channelrhodopsin, also referred to herein as ChR77:

atgggttggaaaatcaatcctctgtactccgatgaggtcgcaatcctggaaatctgtaaagaaaatgaaatggtgtttgggccactg
tgggagcagaagctggcacgggccctccagtggttcactgtcatcctgtcagctatttttctcgcatactacgtgtactccaccctgc
gagcaacatgcggatgggaggaactctatgtctgtactgtggagttcaccaaagtggtcgtggaggtctacctggaatatgtgccc
ccttttatgatctaccagatgaacggccagcacactccttggctgcgatatatggagtggctgctcacctgcccagtgatcctgattc
atctctccaacatcacagggctgaatgacgaatactctggtaggaccatgtctctgctcacaagtgatctgggcggaattgctttcg
cagtcctgagcgccctcgctgtgggatggcagaagggtctgtacttcgggatcggttgcatctacggggccagcaccttctacca
cgccgcttgtatctacatcgagtcataccatactatgcccgctggcaagtgcaaacggctggtcgtggcaatgtgcgccgtgttctt
tacctcttggttcatgtttccagccctgttcctcgctggccccgaatgttttgacggcctgacatggagcggcagcacaatcgcaca
cactgtcgctgatctgctcagcaaaaatatctggggcctgattggacactttctcagagtgggcatccacgagcatattctggtccat
ggagacgtgaggagacctatcgaagtgaccattttcggcaaggagaccagcctgaattgttttgtggagaatgatgatgaggaag
atgatgtc.

SEQ ID NO:6 is the amino acid sequence of wild-type Tetraselmis cordiformis channelrhodopsin, also referred to herein as ChR77:

MGWKINPLYSDEVAILEICKENEMVFGPLWEQKLARALQWFTVILSAIF
LAYYVYSTLRATCGWEELYVCTVEFTKVVVEVYLEYVPPFMIYQMNGQH
TPWLRYMEWLLTCPVILIHLSNITGLNDEYSGRTMSLLTSDLGGIAFAV
LSALAVGWQKGLYFGIGCIYGASTFYHAACIYIESYHTMPAGKCKRLVV
AMCAVFFTSWFMFPALFLAGPECFDGLTWSGSTIAHTVADLLSKNIWGL
IGHFLRVGIHEHILVHGDVRRPIEVTIFGKETSLNCFVENDDEEDDV.

SEQ ID NO:7 is the mammalian codon-optimized DNA sequence that encodes Brachiomonas submarina rhodopsin, also referred to herein as ChR65:

atggaggcctacgcttatccagaactgctggggtctgccggcaggagtctgttcgctgctaccgtgcccgagaacatcagcgaat
ccacatgggtggacgccggctaccagcacttttggactcagagacagaatgagaccgtggtgtgcgaacactatacccatgcca
gctggctgatttcccatggcacaaaggctgagaaaactgccatgatcgcttgtcagtggttcgccttcggcagcgccgtgctgatc
ctgctgctgtacgcctggcacacctggaaggctacatccgggtgggaggaagtgtacgtgtgctgcgtggagctggtgaaagtg
ctgttcgagatctaccatgaaatccaccatccttgcaccctgtatctggtgacaggcaactttattctgtggctgcggtacggagagt
ggctgctgacttgtccagtgattctgatccacctgagcaatattactggactgaagaacgactacaacaagcgcaccatgcagctg
ctggtgtccgatatcggatgcgtggtgtggggagtgacagccgccctgtgctacgactataagaaatggattttcttttgcctgggc
ctggtgtacggatgtaacacctatttccacgccgctaaggtgtatatcgagggatatcatacagtgcccaagggggaatgcaggat
cattgtgaaagtgatggccggcgtgttctactgttcttggacactgttccccctgctgtttctgctggggcctgagggcactggagcc
ttttctgcttatggcagtactatcgcccacaccgtggctgatgtgctgtccaagcagctgtggggactgctggggcaccatctgcgg
gtgaaaattcacgagcatatcattatccacggaaatctgaccgtgtctaagaaagtgaaggtggccggggtggaggtggaaaca
caggaaatggtggacagtactgaggaagatgctgtg.

SEQ ID NO:8 is the amino acid sequence of wild-type Brachiomonas submarina rhodopsin, also referred to herein as ChR65:

MEAYAYPELLGSAGRSLFAATVPENISESTWVDAGYQHFWTQRQNETVV
CEHYTHASWLISHGTKAEKTAMIACQWFAFGSAVLILLLYAWHTWKATS
GWEEVYVCCVELVKVLFEIYHEIHHPCTLYLVTGNFILWLRYGEWLLTC
PVILIHLSNITGLKNDYNKRTMQLLVSDIGCVVWGVTAALCYDYKKWIF
FCLGLVYGCNTYFHAAKVYIEGYHTVPKGECRIIVKVMAGVFYCSWTLF
PLLFLLGPEGTGAFSAYGSTIAHTVADVLSKQLWGLLGHHLRVKIHEHI
IIHGNLTVSKKVKVAGVEVETQEMVDSTEEDAV

SEQ ID NO:9 is the mammalian codon-optimized DNA sequence that encodes the wild-type Channelrhodopsin-2, (see: Boyden, E. et al., Nature Neuroscience 8, 1263-1268 (2005) and Nagel, G., et al. PNAS Nov. 25, 2003 vol. 100 no. 24 13940-13945), also referred to herein as ChR2:

atggactatggcggcgctttgtctgccgtcggacgcgaacttttgttcgttactaatcctgtggtggtgaacgggtccgtcctggtcc
ctgaggatcaatgttactgtgccggatggattgaatctcgcggcacgaacggcgctcagaccgcgtcaaatgtcctgcagtggctt
gcagcaggattcagcattttgctgctgatgttctatgcctaccaaacctggaaatctacatgcggctgggaggagatctatgtgtgc
gccattgaaatggttaaggtgattctcgagttcttttttgagtttaagaatccctctatgctctaccttgccacaggacaccgggtgcag
tggctgcgctatgcagagtggctgctcacttgtcctgtcatccttatccacctgagcaacctcaccggcctgagcaacgactacag
caggagaaccatgggactccttgtctcagacatcgggactatcgtgtggggggctaccagcgccatggcaaccggctatgttaa
agtcatcttcttttgtcttggattgtgctatggcgcgaacacattttttcacgccgccaaagcatatatcgagggttatcatactgtgcca
aagggtcggtgccgccaggtcgtgaccggcatggcatggctgtttttcgtgagctggggtatgttcccaattctcttcattttggggc
ccgaaggttttggcgtcctgagcgtctatggctccaccgtaggtcacacgattattgatctgatgagtaaaaattgttgggggttgtt
gggacactacctgcgcgtcctgatccacgagcacatattgattcacggagatatccgcaaaaccaccaaactgaacatcggcgg
aacggagatcgaggtcgagactctcgtcgaagacgaagccgaggccggagccgtg.

SEQ ID NO: 10 is the amino acid sequence of the wild-type Channelrhodopsin-2, (see: Boyden, E. et al., Nature Neuroscience 8, 1263-1268 (2005) and Nagel, G., et al. PNAS Nov. 25, 2003 vol. 100 no. 24 13940-13945), also referred to herein as ChR2:

MDYGGALSAVGRELLFVTNPVVVNGSVLVPEDQCYCAGWIESRGTNGAQ
TASNVLQWLAAGFSILLLMFYAYQTWKSTCGWEEIYVCAIEMVKVILEF
FFEFKNPSMLYLATGHRVQWLRYAEWLLTCPVILIHLSNLTGLSNDYSR
RTMGLLVSDIGTIVWGATSAMATGYVKVIFFCLGLCYGANTFFHAAKAY
IEGYHTVPKGRCRQVVTGMAWLFFVSWGMFPILFILGPEGFGVLSVYGS
TVGHTIIDLMSKNCWGLLGHYLRVLIHEHILIHGDIRKTTKLNIGGTEI
EVETLVEDEAEAGAV.

SEQ ID NO:11 is the DNA sequence of the ‘ss’ signal sequence from truncated MHC class I antigen:

gtcccgtgcacgctgctcctgctgttggcagccgccctggctccgactc
agacgcgggcc.

SEQ ID NO:12 is the amino acid sequence of the ‘ss’ signal sequence from truncated MHC class I antigen:

MVPCTLLLLLAAALAPTQTRA.

SEQ ID NO:13 is the DNA sequence of the ER export sequence (also referred to herein as ER2″:

ttctgctacgagaatgaagtg.

SEQ ID NO:14 is the amino acid sequence of the ER export sequence (also referred to herein as “ER2”:

FCYENEV.

SEQ ID NO:15 is the DNA sequence of KGC, which is a C terminal export sequence from the potassium channel Kir2.1:

aaatccagaattacttctgaaggggagtatatccctctggatcaaatag
acatcaatgtt.

SEQ ID NO:16 is the amino acid sequence of KGC, which is a C terminal export sequence from the potassium channel Kir2.1:

KSRITSEGEYIPLDQIDINV.

### DETAILED DESCRIPTION

The invention in some aspects relates to the expression in cells of light-driven ion channel polypeptides that can be activated by contact with one or more pulses of light, which results in strong depolarization of the cell. Light-activated channels of the invention, also referred to herein as light-activated ion channels (LAICs) can be expressed in specific cells, tissues, and/or organisms and used to control cells in vivo, ex vivo, and in vitro in response to pulses of light of a suitable wavelength. Ultraviolet light-activated ion channel polypeptides derived from Tetraselmis rhodopsin sequences, have now been identified. In addition, light-activated ion channel polypeptides derived from Brachiomonas rhodopsin sequences, have now been identified and characterized as having little or no calcium ion conductance when activated.

The LAICs of the invention are ion channels and may be expressed in a membrane of a cell. An ion channel is an integral membrane protein that forms a pore through a membrane and assist in establishing and modulating the small voltage gradient that exists across the plasma membrane of all cells and are also found in subcellular membranes of organelles such as the endoplasmic reticulum (ER), mitochondria, etc. When a LAIC of the invention is activated by contacting the cell with appropriate light, the pore opens and permits conductance of ions such as sodium, potassium, calcium, etc. through the pore. It has been identified that certain LAICs of the invention, referred to as low-C LAICs, restrict and/or block conductance of calcium ions conductance, but permit other ions to pass through.

In some embodiments of the invention, light-activated channels may be used to modify the transmembrane potential (and/or ionic composition) of cells (and/or their sub-cellular regions, and their local environment). For example, the use of inwardly rectifying cationic channels will depolarize cells by moving positively charged ions from the extracellular environment to the cytoplasm. Under certain conditions, their use can decrease the intracellular pH (and/or cation concentration) or increase the extracellular pH (and/or cation concentration). In some embodiments, the presence of LAICs in one, two, three, or more (e.g. a plurality) of cells in a tissue or organism, can result in depolarization of the single cell or the plurality of cells by contacting the LAICs with light of suitable wavelength.

When expressed in a cell, some LAICs of the invention can be activated by contacting the cell with ultraviolet light having a wavelength between about 340 nm and 400 nm. These are UV-activated LAICs of the invention, which are referred to herein as UV LAICs. An amino acid sequence motif has been identified in the UV-light-activated ion channel polypeptides. The motif includes specific conserved amino acid residues in the polypeptide sequence of UV LAICs of the invention.

Additionally, another category of LAICs have now been identified. This category of LAICs are activated by light in a range from 385 nm to 530 nm, and permit little or no Ca⁺⁺ ion flow through their channel, these LAICs are referred to herein as “low-C LAICs” herein for low-calcium light-activated ion channels.

### UV-LAICs

Contacting an excitable cell that includes a UV-activated LAIC of the invention with a light in the ultraviolet spectrum strongly depolarizes the cell. For example, contact with light in a wavelength range such as between 340 nm and 350 nm, 350 nm and 360 nm, 360 nm and 370 nm, 370 nm and 380 nm, 380 nm and 390 nm, or 390 nm and 400 nm depolarizes the cell. UV LAICs of the invention have a peak wavelength sensitivity in the UV range and thus demonstrate a higher photocurrent at UV wavelengths than previously identified light-activated channels, for example, ChR2. Exemplary wavelengths of light that may be used to depolarize a cell expressing a UV-light-activated LAIC of the invention, include wavelengths from at least about 340 nm, 345 nm, 350 nm, 355 nm, 360 nm, 365 nm, 370 nm, 375 nm, 380 nm, 385 nm, 390 nm, 395 nm, to about 400 nm, including all wavelengths therebetweeen. UV-light contact can be used to depolarize excitable cells in which one or more UV-light-activated LAICs of the invention are expressed.

### Low-C LAICs

Contacting an excitable cell that includes a low-calcium light-activated ion channel (low-C LAIC) of the invention with a light between about 385 nm and about 530 nm, strongly depolarizes the cell. For example, contact with light in a wavelength range such as between 385 nm and 400 nm, 400 nm and 420 nm, 420 nm and 440 nm, 440 nm and 460 nm, 460 nm and 480 nm, 480 nm and 500 nm, or 500 nm and 530 nm depolarizes the cell. Exemplary wavelengths of light that may be used to depolarize a cell expressing a LAIC of the invention, include wavelengths from at least about 450 nm, 460 nm, 470 nm, 480 nm, 490 nm, 500 nm, 510 nm, 520 nm, to about 530 nm, including all wavelengths therebetweeen. Light contact can be used to depolarize excitable cells in which one or more of this category of light-activated LAICs of the invention is expressed. Another characteristic of low-C LAICs of the invention is that unlike previously identified light activated channels, low-C LAICs of the invention permit effectively zero calcium conductance through the channel when they are activated by light. By “effectively zero” is meant a level may be zero or a level close enough to zero as to be undetectable using standard methods such as a fura2 dye testing method. Methods of Fura2 dye testing are known in the art, see for example: Lin, J Y, et al., Biophys J. 2009 Mar. 4; 96(5):1803-14 and Kleinlogel, S. et al., Nat Neurosci. 2011 April; 14(4):513-8. Epub 2011 Mar. 13, the disclosure of which is incorporated by reference herein. Thus, in context of the invention, “effectively zero” means that an amount, if any, of calcium conductance through an activated low-C LAIC of the invention is at a level so low as to be undetectable at the level of sensitivity of at least one standard detection method, the fura 2 dye testing method. The level of Ca⁺⁺ conductance of an activated low-C LAIC of the invention that is detected using an art-known Ca⁺⁺ conductance testing method may be at least 80%, 85%, 90%, 95%, 96%, 97%, 98%, 99%, or 100% less than the level of Ca⁺⁺ conductance that is detected with the same method in a control, wherein a control may be a cell expressing another light activated ion channel, non-limiting examples of which include ChR2.

Both low-C LAICs and UV LAICs of the invention permit ion conductance and depolarization when contacted under suitable conditions with an appropriate wavelength of light. As will be understood by those in the art, the term “depolarized” used in the context of cells means an upward change in the cell voltage. For example, in an excitable cell at a baseline voltage of about −65 mV, a positive change in voltage, e.g., up to 5, 10, 15, 20, 30, 40, or more millivolts (mV) is a depolarization of that cell. When the change in voltage is sufficient to reach the cell's spike initiation voltage threshold an action potential (e.g. a spike) results. When a cell is depolarized by activating a LAIC of the invention with an appropriate wavelength of light, the cell voltage becomes more positive than the baseline level, and an incoming signal may more easily raise the cell's voltage sufficiently to reach the threshold and trigger an action potential in the cell. It has been discovered that by contacting a cell expressing a UV-light-activated LAIC of the invention with light in the range between about 340 nm to about 400 nm, the voltage of the cell becomes less negative and may rise by at least about 20, 30, 40, 50, 60, 70, 80, 90, 100, mV (depending on the cell type) thus, depolarizing the cell. Similarly, it has been discovered that by contacting a cell expressing a light-activated LAIC of the invention that is activated with light having a wavelength between 385 nm to about 530 nm with light in that range, the voltage of the cell becomes less negative by as much as at least 20, 30, 40, 50, 60, 70, 80, 90, 100, mV, (depending on cell type).

Specific ranges of wavelengths of light useful to activate ion channels of the invention are provided and described herein. It will be understood that a light of appropriate wavelength for activation and will have a power and intensity appropriate for activation. It is well known in the art that light pulse duration, intensity, and power are parameters that can be altered when activating a channel with light. Thus, one skilled in the art will be able to adjust power, intensity appropriately when using a wavelength taught herein to activate a LAIC of the invention. A benefit of a UV LAIC of the invention, may be the ability to “tune” its response using an appropriate illumination variables (e.g., wavelength, intensity, duration, etc.) to activate the channel. Methods of adjusting illumination variables are well-known in the art and representative methods can be found in publications such as: Lin, J., et al., Biophys. J. 2009 Mar. 4; 96(5):1803-14; Wang, H., et al., 2007 Proc Natl Acad Sci USA. 2007 May 8; 104(19):8143-8. Epub 2007 May 1., each of which is incorporated herein by reference. Thus, it is possible to utilize a narrow range of one or more illumination characteristics to activate a LAIC of the invention. This may be useful to illuminate a UV LAIC that is co-expressed with one or more other light activated channels that can be illuminated with a different set of illumination parameters for their activation. Thus, permitting controlled activation of a mixed population of light-activated channels.

In exemplary implementations, the invention comprises methods for preparing and using genes encoding LAICs of the invention that have now been identified. The invention, in part, also includes isolated nucleic acids comprising sequences that encode LAICs of the invention as well as vectors and constructs that comprise such nucleic acid sequences. In some embodiments the invention includes expression of polypeptides encoded by the nucleic acid sequences, in cells, tissues, and organisms.

Not all channelrhodopsins can be expressed in cells and utilized in this fashion, since many do not traffic properly and/or function in mammalian cells. Many channelrhodopsins were screened order to identify Tetraselmis and Brachiomonas rhodopsins as functioning better in mammalian cells than other classes of channelrhodopsins. In addition the Tetraselmis rhodopsins responds strongly to ultra-violet (UV) light, and therefore, since there are other channelrhodopsins that depolarize cells respond to green or yellow light, Tetraselmis rhodopsin can be expressed in a separate population of cells from a population of cells expressing one of these other opsins, allowing multiple colors of light to be used to excite these two populations of cells or neuronal projections from one site, at different times.

It has been identified that UV LAICs of the invention are activated at different wavelengths than previously identified light-activated ion channels. Thus UV LAICs of the invention can be used in either alone, using a selective light spectrum for activation and depolarization and can also be used in combination with other light-activated ion channels that utilize different wavelength of light for activation and depolarization, thus allowing two, three, four, or more different wavelengths of light to be used to depolarize different sets of cells in a tissue or organism by expressing channels with different activation spectra in different cells and then illuminating the tissue and/or organism with the appropriate wavelengths of light to activate the channels and depolarize the cells.

According to some aspects of the invention, a UV-drivable opsin, Tetraselmis striata rhodopsin, may be used. A UV LAIC of the invention can depolarize cells in strong response to UV light with sufficient spectral independence from red-shifted opsins such as VChR1, thus allowing multiple colors of light to be used to depolarize different sets of cells in the same tissue, by expressing channels with different activation spectra genetically in different cells, and then illuminating the tissue with different colors of light. For example, if one set of cells in a tissue (e.g., excitatory neurons) express VChR1, and a second set express a UV LAIC of the invention, then illuminating the tissue with 590 nm light will preferentially depolarize the first set, whereas illuminating the tissue with 365 nm light will preferentially depolarize the second set. Other pairs of targets that could be modulated with two colors of light in the same illumination area include, but are not limited to two projections to/from one site, or combinations of the cell, its projections, and its organelles, given the ability to target the molecule sub-cellularly.

### Taxonomy and Sequence Sources

In particular, the present invention includes, in part, novel LAICs and their use to depolarize cells. In some non-limiting embodiments of the invention one or more newly identified LAIC may be expressed in cells.

Some UV-LAICs of the invention have amino acid sequences derived from Tetraselmis rhodopsins that are naturally expressed in the genus Tetraselmis striata, Tetraselmis chui, Tetraselmis cordiformis, or another member of the Chlamydomonadaceae family. Tetraselmis striata, Tetraselmis chuii, Tetraselmis cordiformis, and other members of the Chlamydomonadaceae family are phytoplankton and can be found in fresh or salt water environments. Some embodiments of the invention include isolated wild-type or modified nucleic acid and/or amino acid channelrhodopsin sequences from a member of the chlamydomonadaceae family, for example, from tetraselmis straita, tetraselmis chui, or other genus of chlamydomonadaceae, and in some aspects, the invention also includes methods for their use.

Sequences of UV LAICs of the invention, may be, but are not necessarily derived from a Tetraselmis sequence, and UV LAICs of the invention also be include a wild-type or modified channelrhodopsin sequence from another organism. ChR2 is not an LAIC of the invention, but sequences having homology to the ChR2 sequence set forth herein as SEQ ID NO:10, but that have conserved amino acids at positions that correspond to amino acids 147, 151, 154, 159, and 187 numbered in reference to the sequence of ChR2 set forth as SEQ ID NO:10. The conserved amino acids are conserved in the UV LAICs of the invention differ from ChR2 at each of the ChR2-referenced positions 147, 151, 154, 159, and 187. In the ChR2 the amino acids at positions 147, 151, 154, 159, and 187 are as follows: R147, G151, V154, T159, N187 (numbers in reference to the ChR2 amino acid sequence provided herein). The amino acids in the corresponding positions in UV LAIC sequences have been identified as G147, 5151, T154, G159, 5187 [amino acid numbered in reference to ChR2 amino acid sequence provided herein as SEQ ID NO:10)].

It has now been identified that in UV LAICs of the invention, the amino acids at the positions corresponding to amino acids 147, 151, 154, 159, and 187 are conserved in UV LAICs of the invention. The conserved amino acids have been identified as a “motif” for the UV light-activated ion channels of the invention. Additional channelrhodopsin sequences that are activated by UV light and/or include a motif of these conserved amino acid residues, may be considered UV LAICs of the invention and based on the disclosure and sequences provided herein, can be identified and utilized in methods of the invention.

The conserved amino acids in the ChR66 sequence are positioned in the ChR66 sequence as follows: G129, 5133, T136, G141, and 5169, numbered in reference to the sequence of ChR66 set forth herein as SEQ ID NO:2. The conserved amino acids in the ChR68 sequence are as follows: G131, 5135, T138, G143, and 5171, numbered in reference to the sequence of ChR68 set forth herein as SEQ ID NO:4. The conserved amino acid in the ChR77 UV LAIC sequence are: G130, 5134, T137, G142, 5170 numbered in reference to the amino acid sequence of ChR77 set forth herein as SEQ ID NO:6.

One skilled in the art will understand that UV LAICs of the invention can be identified based on sequence homology to a UV LAIC disclosed herein. It will be understood that additional LAICs may be identified using sequence alignment with ChR2, plus the presence of the conserved residues, although homology will likely not be as high with ChR2 as with one or more of the UV LAIC sequences disclosed herein.

Based on the teaching provided herein regarding the Tetraselmis channelrhodopsin sequences having UV LAIC function and activity, additional rhodopsin sequences with sufficient amino acid sequence homology to a Tetraselmis rhodopsin sequence such as ChR66, ChR68, ChR77, set forth herein, can be identified. Such a UV LAIC would have homology to a ChR66, ChR68, ChR77 (or ChR2) sequence and would include the corresponding conserved amino acid residues G129, 5133, T136, G141, and 5169, (numbered in reference to the amino acid sequence of ChR66 set forth herein as SEQ ID NO:2). If a sequence were aligned with ChR2, corresponding conserved amino acid residues G147, S151, T154, G159, S187 (numbered in reference to ChR2 amino acid sequence set forth herein as SEQ ID NO:10), and that are activated by UV light, can be identified as a UV LAIC, and can be used in methods of the invention. It will be clear that homology with other LAICs of the invention plus conservation of the five specified amino acids, plus UV activation can be characteristics used to identify additional LAICs using standard procedures for sequence alignment, comparisons, and assays for ion channel activity. The locations of the conserved amino acids (the motif) in relation to each sequence are shown in Table 1. Chr2 is not an LAIC of the invention and does not include the conserved amino acids at the requisite corresponding positions. Table 1 shows the conserved amino acids at their corresponding locations in ChR66, ChR68, ChR77, and the position in ChR2, showing the amino acids at the locations in ChR2, as outlined above.

TABLE 1
Corresponding motif amino acids and relative positions in
ChR2 and in exemplary LAIC sequences of the invention.

| | AA ID | AA ID | AA ID | AA ID | AA ID |
|---|---|---|---|---|---|
| | and | and | and | and | and |
| Ion Channel | Motif | Motif | Motif | Motif | Motif |
| Name | Position | Position | Position | Position | Position |
| ChR2 | R147 | G151 | V154 | T159 | N187 |
| SEQ ID NO: 10 | | | | | |
| ChR66 | G129 | S133 | T136 | G141 | S169 |
| SEQ ID NO: 2 | | | | | |
| ChR68 | G131 | S135 | T138 | G143 | S171 |
| SEQ ID NO: 4 | | | | | |
| ChR77 | G130 | S134 | T137 | G142 | S170 |
| SEQ ID NO: 6 | | | | | |

AA ID = amino acid identity.

Some low-C LAICs of the invention have amino acid sequences derived from Brachiomonas rhodopsins that may be naturally expressed in the genus Brachiomonas simplex, Brachiomonas submarina, and/or Brachiomonas westiana. Brachiomonas are green algae and are found in marine or brackish waters and some freshwater environments. Some embodiments of the invention include isolated wild-type or modified nucleic acid and/or amino acid channelrhodopsin sequences from a member of the genus Brachiomonas, and in some aspects, the invention also includes methods for their use. An exemplary low-C LAIC of the invention is ChR65.

Sequences of all low-C LAICs of the invention, may be, but are not necessarily derived from a Brachiomonas sequence, and also include wild-type of modified channelrhodopsin sequences from other organisms, wherein the channelrhodopsin functions as light-activated ion channels and has essentially zero calcium ion conductance when activated. Based on the teaching provided herein regarding Brachiomonas channelrhodopsin sequences having low-C LAIC function and activity, additional rhodopsin sequences with sufficient amino acid sequence homology to a Brachiomonas rhodopsin sequences such as ChR65, can be identified as low-C LAICs and can be used in methods of the invention.

LAICs of the invention are transmembrane channel polypeptides that use light energy open permitting ion conductance through their pore, thus altering the potential of the membrane in which they are expressed. A non-limiting example of an ion that can be moved through a pore of the invention includes a sodium ion, a potassium ion, a proton, etc. In the case of tetraselmis, calcium ions may also be moved through a pore, but as described elsewhere herein, little or no calcium can be passed through a brachiomonas LAIC of the invention. LAICs of the invention can be activated by sustained light and/or by light pulses and by permitting ion conductance upon activation, LAICs of the invention can depolarize cells and alter the voltage in cells and organelles in which they are expressed.

The wild-type and modified Tetraselmis and Brachiomonas rhodopsin nucleic acid and amino acid sequences used in aspects and methods of the invention are “isolated” sequences. As used herein, the term “isolated” used in reference to a polynucleotide, nucleic acid sequence or polypeptide sequence of a rhodopsin, it means a polynucleotide, nucleic acid sequence, or polypeptide sequence that is separate from its native environment and present in sufficient quantity to permit its identification or use. Thus, an isolated polynucleotide, nucleic acid sequence, or polypeptide sequence of the invention is a polynucleotide, nucleic acid sequence, or polypeptide sequence that is not part of, or included in its native host. For example, a nucleic acid or polypeptide sequence may be naturally expressed in a cell or organism of a member of the Tetraselmis genus, but when the sequence is not part of or included in a Tetraselmis cell or organism, it is considered to be isolated. Similarly, a nucleic acid or polypeptide sequence may be naturally expressed in a cell or organism of a member of the Brachiomonas genus, but the sequence is not part of or included in a Brachiomonas cell or organism, it is considered to be isolated. Thus, a nucleic acid or polypeptide sequence of a Tetraselmis, Brachiomonas, or other channelrhodopsin that is present in a vector, in a heterologous cell, tissue, or organism, etc., is an isolated sequence. The term “heterologous” as used herein, means a cell, tissue or organism that is not the native cell, tissue, or organism. The terms, “protein”, “polypeptides”, and “peptides” are used interchangeably herein.

### LAIC Sequences Including Modified Sequences

A LAIC of the invention may comprise a wild-type polypeptide sequence or may be a modified polypeptide sequence. As used herein the term “modified” or “modification” in reference to a nucleic acid or polypeptide sequence refers to a change of one, two, three, four, five, six, or more amino acids in the sequence as compared to the wild-type sequence from which it was derived. For example, a modified polypeptide sequence may be identical to a wild-type polypeptide sequence except that it has one, two, three, four, five, or more amino acid substitutions, deletions, insertions, or combinations thereof. In some embodiments of the invention a modified sequence may include one, two, three, four, or more amino acid substitutions in a wild-type channelrhodopsin sequence.

It will be understood that sequences of LAICs of the invention may be derived from various members of the Tetraselmis genus, the Brachiomonas genus or from other channelrhodopsin sequences that may correspond, at least in part, to a Tetraselmis or Brachiomonas sequence disclosed herein. For example, SEQ ID NO:2 is the wild-type amino acid sequence of the Tetraselmis striata channelrhodopsin polypeptide referred to herein as ChR66. SEQ ID NO:4 is the wild-type amino acid sequence of the Tetraselmis chuii channelrhodopsin polypeptide referred to herein as ChR68. SEQ ID NO:6 is the wild-type amino acid sequence of the Tetraselmis cordiformis channelrhodopsin polypeptide referred to herein as ChR77. SEQ ID NO:8 is the wild-type amino acid sequence of the Brachiomonas channelrhodopsin polypeptide referred to herein as ChR65. Using standard methods for determining sequence homology one of ordinary skill in the art is able to identify additional channelrhodopsin sequences (including, but not limited to other phytoplankton or algal channelrhodopsin sequences) to identify homologous polypeptides that also function as LAICs of the invention. Also, using standard sequence search and/or alignment methods, one can readily identify channelrhodopsin sequences that include a motif of conserved amino acid residues G 147, S151, T154, G159, S187 [amino acid are numbered in reference to ChR2 amino acid sequence provided herein as SEQ ID NO:10)] and function as UV LAICs of the invention.

The invention, in some aspects also includes LAIC polypeptides having one or more substitutions or other modifications from those described herein. For example, sequences of LAIC polypeptides can be modified with one or more substitutions, deletions, insertions, or other modifications and can be tested using methods described herein for characteristics including, but not limited to: expression, cell localization, activation and depolarization in response to contact with light using methods disclosed herein. Exemplary modifications include, but are not limited to conservative amino acid substitutions, which will produce molecules having functional characteristics similar to those of the molecule from which such modifications are made. Conservative amino acid substitutions” are substitutions that do not result in a significant change in the activity or tertiary structure of a selected polypeptide or protein. Such substitutions typically involve replacing a selected amino acid residue with a different residue having similar physico-chemical properties. For example, substitution of Glu for Asp is considered a conservative substitution because both are similarly-sized negatively-charged amino acids. Groupings of amino acids by physico-chemical properties are known to those of skill in the art. The following groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). LAICs that include modifications, including but not limited to one, two, three, four, or more conservative amino acid substitutions can be identified and tested for characteristics including, but not limited to: expression, cell localization, activation and depolarization and depolarization-effects in response to contact with light using methods disclosed herein.

A LAIC polypeptide of the invention may include amino acid variants (e.g., polypeptides having a modified sequence) of a sequence set forth herein or another rhodopsin sequence. Modified LAIC polypeptide sequences may be at least about 75%, 80%, 85%, 90%, 95%, 96%, 97%, 98%, 99%, or 100% homologous to the polypeptide sequence of a LAIC disclosed herein, such as ChR66, ChR68, ChR77, ChR65, etc. Homology in this context means sequence similarity or identity. Such sequence homology can be determined using standard techniques known in the art. LAICs of the present invention include the LAIC polypeptide and nucleic acid sequences provided herein and variants that are more than about 60%, 65%, 70%, 75%, 80%, 85%, 90%, 95%, 96%, 97%, or 98% homologous to a provided sequence.

LAIC polypeptides of the invention may be shorter or longer than the LAIC polypeptide sequences set forth herein. Thus, in a preferred embodiment, included within the definition of LAIC polypeptides are full-length polypeptides or functional fragments thereof. In addition, nucleic acids of the invention may be used to obtain additional coding regions, and thus additional polypeptide sequences, using techniques known in the art.

In some aspects of the invention, substantially similar LAIC polypeptide sequences may have at least 80%, 85%, 90%, 91%, 92%, 93%, 94%, 95%, 96%, 97%, 98%, or 99% or 100% similarity to a LAIC sequence disclosed herein, non-limiting examples of which include as ChR66, ChR68, ChR77, ChR65, etc. Art-known alignment methods and tools can be used to align substantially similar sequences permitting positional identification of amino acids that may be modified as described herein to prepare a LAIC of the invention. Standard sequence analysis tools and computer programs, such as those used for alignment, etc. can be used to identify channelrhodopsin sequences that include the motif of conserved amino acids G147, S151, T154, G159, S187 [amino acid are numbered in reference to ChR2 amino acid sequence provided herein as SEQ ID NO:10)] and are activated by UV light. Similarly, art-known sequence tools and programs can be used to identify channelrhodopsin sequences that have a sequence homology to a low-C LAIC of the invention and that share similar functional properties with a low-C LAIC described herein.

Sequence modifications can be in one or more of three classes: substitutions, insertions, or deletions. These modified sequences, (which may also be referred to as variants) ordinarily are prepared by site specific mutagenesis of nucleic acids in the DNA encoding a LAIC polypeptide, using cassette or PCR mutagenesis or other techniques known in the art, to produce DNA encoding the modified LAIC, and thereafter expressing the DNA in recombinant cell culture. Amino acid sequence variants are characterized by the predetermined nature of the variation, a feature that sets them apart from naturally occurring allelic or interspecies variation of the LAICs of the invention. Modified LAICs generally exhibit the same qualitative biological activity as the naturally occurring analogue, although variants can also be selected that have modified characteristics.

A site or region for introducing an amino acid sequence modification may be predetermined, and the mutation per se need not be predetermined. For example, to optimize the performance of a mutation at a given site, random mutagenesis may be conducted at the target codon or region and the expressed modified LAIC screened for the optimal combination of desired activity. Techniques for making substitution mutations at predetermined sites in DNA having a known sequence are well known, for example, M13 primer mutagenesis and PCR mutagenesis.

Amino acid substitutions are typically of single residues; and insertions usually will be on the order of from about 1 to 20 amino acids, although considerably larger insertions may be tolerated. Deletions may range from about 1 to about 20 residues, although in some cases deletions may be much larger.

Substitutions, deletions, insertions or any combination thereof may be used to arrive at a final modified LAIC of the invention. Generally these changes are done on a few amino acids to minimize the alteration of the molecule. However, larger changes may be tolerated in certain circumstances.

Variants of LAICs set forth herein, may exhibit the same qualitative light-activated ion channel activity as one or more of the sequences set forth herein, such as ChR66, ChR68, ChR77, or ChR65, but may show some altered characteristics such as altered photocurrent, stability, speed, compatibility, and toxicity, or a combination thereof. For example, the polypeptide can be modified such that it has an increased photocurrent and/or has less toxicity than another LAIC polypeptide.

A modified LAIC polypeptide of the invention can incorporate unnatural amino acids as well as natural amino acids. An unnatural amino acid can be included in a LAIC of the invention to enhance a characteristic such as photocurrent, stability, speed, compatibility, or to lower toxicity, etc.

According to principles of this invention, the performance of LAIC molecules or classes of molecules can be tuned for optimal use, including in the context of their use in conjunction with other molecules or optical apparatus. For example, in order to achieve optimal contrast for multiple-color stimulation, one may desire to either improve or decrease the performance of one molecule with respect to one another, by the appendage of trafficking enhancing sequences or creation of genetic variants by site-directed mutagenesis, directed evolution, gene shuffling, or altering codon usage. LAIC molecules or classes of molecules may have inherently varying spectral sensitivity. This may be used to advantage in vivo (where scattering and absorption will vary with respect to wavelength, coherence, and polarization), by tuning the linearity or non-linearity of response to optical illumination with respect to time, power, and illumination history.

In some embodiments, the invention includes the use of targeted site-directed mutagenesis at specific amino acid residues of channelrhodopsins including but not limited to residues of channelrhodopsins of Tetraselmis and Brachiomonas. Specific locations for single mutations can be identified and alone, or in combination with two or more additional mutations can be placed into a channelrhodopsin sequence and tested with respect to their activation and photocurrent amplitude. Thus, sequences of LAICs of the invention, and/or similar channelrhodopsin sequences can be modified and the resulting polypeptides tested using methods disclosed herein.

Another aspect of the invention provides nucleic acid sequences that code for a LAIC of the invention. It would be understood by a person of skill in the art that the LAIC polypeptides of the present invention can be coded for by various nucleic acids. Each amino acid in the protein is represented by one or more sets of 3 nucleic acids (codons). Because many amino acids are represented by more than one codon, there is not a unique nucleic acid sequence that codes for a given protein. It is well understood by those of skill in the art how to make a nucleic acid that can code for LAIC polypeptides of the invention by knowing the amino acid sequence of the protein. A nucleic acid sequence that codes for a polypeptide or protein is the “gene” of that polypeptide or protein. A gene can be RNA, DNA, or other nucleic acid than will code for the polypeptide or protein.

It is understood in the art that the codon systems in different organisms can be slightly different, and that therefore where the expression of a given protein from a given organism is desired, the nucleic acid sequence can be modified for expression within that organism. Thus, in some embodiments, a LAIC polypeptide of the invention is encoded by a mammalian-codon-optimized nucleic acid sequence, which may in some embodiments be a human-codon optimized nucleic acid sequence. An aspect of the invention provides a nucleic acid sequence that codes for a LAIC that is optimized for expression with a mammalian cell. A preferred embodiment comprises a nucleic acid sequence optimized for expression in a human cell.

### Delivery of LAICs

Delivery of a LAIC polypeptide to a cell and/or expression of a LAIC in a cell can be done using art-known delivery means.

In some embodiments of the invention a LAIC polypeptide of the invention is included in a fusion protein. It is well known in the art how to prepare and utilize fusion proteins that comprise a polypeptide sequence. In certain embodiments of the invention, a fusion protein can be used to deliver a LAIC to a cell and can also in some embodiments be used to target a LAIC of the invention to specific cells or to specific cells, tissues, or regions in a subject. Targeting and suitable targeting sequences for deliver to a desired cell, tissue or region can be performed using art-known procedures.

It is an aspect of the invention to provide a LAIC polypeptide of the invention that is non-toxic, or substantially non-toxic in cells in which it is expressed. In the absence of light, a LAID of the invention does not significantly alter cell health or ongoing electrical activity in the cell in which it is expressed.

In some embodiments of the invention, a LAIC of the invention is genetically introduced into a cellular membrane, and reagents and methods are provided for genetically targeted expression of LAIC polypeptides, including ChR66, ChR68, ChR77, ChR65, etc. Genetic targeting can be used to deliver LAIC polypeptides to specific cell types, to specific cell subtypes, to specific spatial regions within an organism, and to sub-cellular regions within a cell. Genetic targeting also relates to the control of the amount of LAIC polypeptide expressed, and the timing of the expression.

Some embodiments of the invention include a reagent for genetically targeted expression of a LAIC polypeptide, wherein the reagent comprises a vector that contains the gene for the LAIC polypeptide.

As used herein, the term “vector” refers to a nucleic acid molecule capable of transporting between different genetic environments another nucleic acid to which it has been operatively linked. The term “vector” also refers to a virus or organism that is capable of transporting the nucleic acid molecule. One type of vector is an episome, i.e., a nucleic acid molecule capable of extra-chromosomal replication. Some useful vectors are those capable of autonomous replication and/or expression of nucleic acids to which they are linked. Vectors capable of directing the expression of genes to which they are operatively linked are referred to herein as “expression vectors”. Other useful vectors, include, but are not limited to viruses such as lentiviruses, retroviruses, adenoviruses, and phages. Vectors useful in some methods of the invention can genetically insert LAIC polypeptides into dividing and non-dividing cells and can insert LAIC polypeptides to cells that are in vivo, in vitro, or ex vivo cells.

Vectors useful in methods of the invention may include additional sequences including, but not limited to one or more signal sequences and/or promoter sequences, or a combination thereof. Expression vectors and methods of their use are well known in the art. Non-limiting examples of suitable expression vectors and methods for their use are provided herein.

In certain embodiments of the invention, a vector may be a lentivirus comprising the gene for a LAIC of the invention, such as ChR66, ChR68, ChR77, ChR65, or a variant thereof. A lentivirus is a non-limiting example of a vector that may be used to create stable cell line. The term “cell line” as used herein is an established cell culture that will continue to proliferate given the appropriate medium.

Promoters that may be used in methods and vectors of the invention include, but are not limited to, cell-specific promoters or general promoters. Methods for selecting and using cell-specific promoters and general promoters are well known in the art. A non-limiting example of a general purpose promoter that allows expression of a LAIC polypeptide in a wide variety of cell types—thus a promoter for a gene that is widely expressed in a variety of cell types, for example a “housekeeping gene” can be used to express a LAIC polypeptide in a variety of cell types. Non-limiting examples of general promoters are provided elsewhere herein and suitable alternative promoters are well known in the art.

In certain embodiments of the invention, a promoter may be an inducible promoter, examples of which include, but are not limited to tetracycline-on or tetracycline-off, or tamoxifen-inducible Cre-ER.

### Methods of Use of LAICs of the Invention

LAICs of the invention are well suited for targeting cells and specifically altering voltage-associated cell activities. In some embodiments of the invention, LAICs of the invention can utilized to introduce cations into cells, thus activating endogenous signaling pathways (such as calcium dependent signaling), and then drugs are applied that modulate the response of the cell (using a calcium or voltage-sensitive dye). This allows new kinds of drug screening using just light to activate the channels of interest, and using just light to read out the effects of a drug on the channels of interest.

In certain aspects of the invention, a Tetraselmis LAIC of the invention may be used to sensitize cells to ultraviolet light. Such methods may be used to treat blindness and introduce visual perception to non-visible light.

In another aspect of the invention, a LAIC may be used to decrease the pH of a cell in which it is expressed. Such a technique may be used to treat alkalosis.

Another aspect of the invention includes methods of using light-activated proton pumps in conjunction with the use of LAICs of the invention for the coupled effect of hyperpolarization and intracellular alkalinization. For example, both phenomena can induce spontaneous spiking in neurons by triggering hyperpolarization-induced cation currents or pH-dependent hyper-excitability.

Another aspect of the invention includes a specific use of the Tetraselmis genus of chlorophyte. These are efficacious UV-activated ion channels and they express particularly well in mammalian membranes and perform robustly under mammalian physiological conditions. Another aspect of the invention is to generate sub-cellular voltage or pH gradients, particularly at synapses and in synaptic vesicles to alter synaptic transmission, and mitochondria to improve ATP synthesis.

Working operation of a prototype of this invention was demonstrated by genetically expressing light-activated ion channel molecules of the invention in excitable cells, illuminating the cells with suitable wavelengths of light, and demonstrating rapid depolarization of the cells in response to the light, as well as rapid release from depolarization upon cessation of light. Depending on the particular implementation, methods of the invention allow light control of cellular functions in vivo, ex vivo, and in vitro.

In non-limiting examples of methods of the invention, microbial channelrhodopsins are used in mammalian cells without need for any kind of chemical supplement, and in normal cellular environmental conditions and ionic concentrations. For example, genes encoding channelrhodopsins of Tetraselmis and Brachiomonas have been used in exemplary implementations of the invention. These sequences in humanized or mouse-optimized form allow depolarization at UV wavelengths (e.g., UV LAICs of the invention) or depolarization with essentially zero calcium conductance (e.g., low-C LAICS of the invention).

As used herein, the term “ion channel” means a transmembrane polypeptide that forms a pore, which when activated opens, permitting ion conductance through the pore across the membrane. Many ion channels do not express well in a cell and/or their expression may be toxic to the cell and reduce cell health. Thus it was necessary to prepare and screen numerous channelrhodopsin light-activated ion channel polypeptides to identify light-activated ion channels of the invention that can be expressed in cells without significantly reducing cell health and viability.

Light-activated ion channels of the invention have been found to be suitable for expression and use in mammalian cells without need for any kind of chemical supplement, and in normal cellular environmental conditions and ionic concentrations. LAICs of the invention have been found to differ from previously identified channels in that the UV LAICs activate at a wavelengths of light in the ultraviolet range, e.g., from 340 to 400 nm and that the low-C LAICs of the invention, when activated by light, permit little or no calcium conductance.

### Cells and Subjects

A cell used in methods and with sequences of the invention may be an excitable cell or a non-excitable cell. A cell in which a LAIC of the invention may be expressed and may be used in methods of the invention include prokaryotic and eukaryotic cells. Useful cells include but are not limited to mammalian cells. Examples of cells in which a LAIC of the invention may be expressed are excitable cells, which include cells able to produce and respond to electrical signals. Examples of excitable cell types include, but are not limited to neurons, muscles, cardiac cells, and secretory cells (such as pancreatic cells, adrenal medulla cells, pituitary cells, etc.).

Non-limiting examples of cells that may be used in methods of the invention include: nervous system cells, cardiac cells, circulatory system cells, visual system cells, auditory system cells, secretory cells, endocrine cells, or muscle cells. In some embodiments, a cell used in conjunction with the invention may be a healthy normal cell, which is not known to have a disease, disorder or abnormal condition. In some embodiments, a cell used in conjunction with methods and channels of the invention may be an abnormal cell, for example, a cell that has been diagnosed as having a disorder, disease, or condition, including, but not limited to a degenerative cell, a neurological disease-bearing cell, a cell model of a disease or condition, an injured cell, etc. In some embodiments of the invention, a cell may be a control cell.

LAICs of the invention may be expressed in cells from culture, cells in solution, cells obtained from subjects, and/or cells in a subject (in vivo cells). LAICs may be expressed and activated in cultured cells, cultured tissues (e.g., brain slice preparations, etc.), and in living subjects, etc. As used herein, a the term “subject” may refer to a human, non-human primate, cow, horse, pig, sheep, goat, dog, cat, rodent, fly or any other vertebrate or invertebrate organism.

### Controls and Candidate Compound Testing

LAICs of the invention and methods using LAICs of the invention can be utilized to assess changes in cells, tissues, and subjects in which they are expressed. Some embodiments of the invention include use of LAICs of the invention to identify effects of candidate compounds on cells, tissues, and subjects. Results of testing a LAIC of the invention can be advantageously compared to a control.

As used herein a control may be a predetermined value, which can take a variety of forms. It can be a single cut-off value, such as a median or mean. It can be established based upon comparative groups, such as cells or tissues that include the LAIC and are contacted with light, but are not contacted with the candidate compound and the same type of cells or tissues that under the same testing condition are contacted with the candidate compound. Another example of comparative groups may include cells or tissues that have a disorder or condition and groups without the disorder or condition. Another comparative group may be cells from a group with a family history of a disease or condition and cells from a group without such a family history. A predetermined value can be arranged, for example, where a tested population is divided equally (or unequally) into groups based on results of testing. Those skilled in the art are able to select appropriate control groups and values for use in comparative methods of the invention.

As a non-limiting example of use of a LAIC to identify a candidate therapeutic agent or compound, a LAIC of the invention may be expressed in an excitable cell in culture or in a subject and the excitable cell may be contacted with a light that activates the LAIC and with a candidate therapeutic compound. In one embodiment, a test cell that includes a LAIC of the invention can be contacted with a light that depolarizes the cell and also contacted with a candidate compound. The cell, tissue, and/or subject that include the cell can be monitored for the presence or absence of a change that occurs in the test conditions versus the control conditions. For example, in a cell, a change may be a change in the depolarization or in a depolarization-mediated cell characteristic in the test cell versus a control cell, and a change in depolarization or the depolarization-mediated cell characteristic in the test cell compared to the control may indicate that the candidate compound has an effect on the test cell or tissue that includes the cell. In some embodiments of the invention, a depolarization-mediated cell characteristic may be a an action potential, pH change in a cell, release of a neurotransmitter, etc. and may in come embodiments, include a downstream effect on one or more additional cells, which occurs due to the depolarization of the cell that includes the LAIC. Art-known methods can be sued to assess depolarization and depolarization-mediated cell characteristics and changes to the depolarization or depolarization-mediated cell characteristics upon excitation of a LAIC of the invention, with or without additional contact with a candidate compound.

Candidate-compound identification methods of the invention that are performed in a subject, may include expressing a LAIC in the subject, contacting the subject with a light under suitable conditions to activate the LAIC and depolarize the cell, and administering to the subject a candidate compound. The subject is then monitored to determine whether any change occurs that differs from a control effect in a subject. Thus, for example, a cell in culture can be contacted with a light appropriate to activate a LAIC of the invention in the presence of a candidate compound. A result of such contact with the candidate compound can be measured and compared to a control value as a determination of the presence or absence of an effect of the candidate compound.

Methods of identifying effects of candidate compounds using LAICs of the invention may also include additional steps and assays to further characterizing an identified change in the cell, tissue, or subject when the cell is contacted with the candidate compound. In some embodiments, testing in a cell, tissue, or subject can also include one or more cells that has a LAIC of the invention, and that also has one, two, three, or more additional different light-activated ion channels, wherein at least one, two, three, four, or more of the additional light-activated ion channels is activated by contact with light having a different wavelength than used to activate the UV LAIC or low-C LAIC of the invention.

In a non-limiting example of a candidate drug identification method of the invention, cells that include a LAIC of the invention are depolarized, thus triggering release of a neurotransmitter from the cell, and then drugs are applied that modulate the response of the cell to depolarization (determined for example using patch clamping methods or other suitable art-known means). Such methods enable new kinds of drug screening using just light to activate the channels of interest, and using just light to read out the effects of a drug on the channels and channel-containing cells of interest.

In some embodiments, LAIC polypeptides of the invention can be used in test systems and assays for assessing membrane protein trafficking and physiological function in heterologously expressed systems and the use of use of light-activated channels to depolarize a cell.

### Methods of Treating

Some aspects of the invention include methods of treating a disorder or condition in a cell, tissue, or subject using LAICs of the invention. Treatment methods of the invention may include administering to a subject in need of such treatment, a therapeutically effective amount of a LAIC of the invention to treat the disorder. It will be understood that a treatment may be a prophylactic treatment or may be a treatment administered following the diagnosis of a disease or condition. A treatment of the invention may reduce or eliminate a symptom or characteristic of a disorder, disease, or condition or may eliminate the disorder, disease, or condition itself. It will be understood that a treatment of the invention may reduce or eliminate progression of a disease, disorder or condition and may in some instances result in the regression of the disease, disorder, or condition. A treatment need to entirely eliminate the disease, disorder, or condition to be effective.

Administration of a LAIC of the invention may include administration pharmaceutical composition that includes a cell, wherein the cell expresses the light-activated ion channel. Administration of a LAIC of the invention may include administration of a pharmaceutical composition that includes a vector, wherein the vector comprises a nucleic acid sequence encoding the light-activated ion channel and the administration of the vector results in expression of the light-activated ion channel in a cell in the subject.

An effective amount of a LAIC is an amount that increases LAIC in a cell, tissue or subject to a level that is beneficial for the subject. An effective amount may also be determined by assessing physiological effects of administration on a cell or subject, such as a decrease in symptoms following administration. Other assays will be known to one of ordinary skill in the art and can be employed for measuring the level of the response to a treatment. The amount of a treatment may be varied for example by increasing or decreasing the amount of the LAIC administered, by changing the therapeutic composition in which the LAIC is administered, by changing the route of administration, by changing the dosage timing, by changing the activation amounts and parameters of a LAIC of the invention, and so on. The effective amount will vary with the particular condition being treated, the age and physical condition of the subject being treated; the severity of the condition, the duration of the treatment, the nature of the concurrent therapy (if any), the specific route of administration, and the like factors within the knowledge and expertise of the health practitioner. For example, an effective amount may depend upon the location and number of cells in the subject in which the LAIC is to be expressed. An effective amount may also depend on the location of the tissue to be treated.

Effective amounts will also depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of a composition to increase the level of a LAIC, and/or to alter the length or timing of activation of a LAIC in a subject (alone or in combination with other therapeutic agents) be used, that is, the highest safe dose or amount according to sound medical judgment. It will be understood by those of ordinary skill in the art, however, that a patient may insist upon a lower dose or tolerable dose for medical reasons, psychological reasons or for virtually any other reasons.

A LAIC of the invention may be administered using art-known methods. The manner and dosage administered may be adjusted by the individual physician or veterinarian, particularly in the event of any complication. The absolute amount administered will depend upon a variety of factors, including the material selected for administration, whether the administration is in single or multiple doses, and individual subject parameters including age, physical condition, size, weight, and the stage of the disease or condition. These factors are well known to those of ordinary skill in the art and can be addressed with no more than routine experimentation.

Pharmaceutical compositions that deliver LAICs of the invention may be administered alone, in combination with each other, and/or in combination with other drug therapies, or other treatment regimens that are administered to subjects. A pharmaceutical composition used in the foregoing methods preferably contain an effective amount of a therapeutic compound that will increase the level of a LAIC polypeptide to a level that produces the desired response in a unit of weight or volume suitable for administration to a subject.

The dose of a pharmaceutical composition that is administered to a subject to increase the level of LAIC in cells of the subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits. The amount and timing of activation of a LAIC of the invention (e.g., light wavelength, length of light contact, etc.) that has been administered to a subject can also be adjusted based on efficacy of the treatment in a particular subject. Parameters for illumination and activation of LAICs that have been administered to a subject can be determined using art-known methods and without requiring undue experimentation.

Various modes of administration will be known to one of ordinary skill in the art that can be used to effectively deliver a pharmaceutical composition to increase the level of LAIC in a desired cell, tissue or body region of a subject. Methods for administering such a composition or other pharmaceutical compound of the invention may be topical, intravenous, oral, intracavity, intrathecal, intrasynovial, buccal, sublingual, intranasal, transdermal, intravitreal, subcutaneous, intramuscular and intradermal administration. The invention is not limited by the particular modes of administration disclosed herein. Standard references in the art (e.g., Remington's Pharmaceutical Sciences, 18th edition, 1990) provide modes of administration and formulations for delivery of various pharmaceutical preparations and formulations in pharmaceutical carriers. Other protocols which are useful for the administration of a therapeutic compound of the invention will be known to one of ordinary skill in the art, in which the dose amount, schedule of administration, sites of administration, mode of administration (e.g., intra-organ) and the like vary from those presented herein.

Administration of a cell or vector to increase LAIC levels in a mammal other than a human; and administration and use of LAICs of the invention, e.g. for testing purposes or veterinary therapeutic purposes, is carried out under substantially the same conditions as described above. It will be understood by one of ordinary skill in the art that this invention is applicable to both human and animals. Thus this invention is intended to be used in husbandry and veterinary medicine as well as in human therapeutics.

In some aspects of the invention, methods of treatment using a LAIC of the invention are applied to cells including but not limited to a nervous system cell, a neuron, a cardiac cell, a circulatory system cell, a visual system cell, an auditory system cell, a muscle cell, or an endocrine cell, etc. Disorders and conditions that may be treated using methods of the invention include, injury, brain damage, degenerative neurological conditions (e.g., Parkinson's disease, Alzheimer's disease, seizure, vision loss, hearing loss, etc.

### Disorders, Diseases and Conditions

LAICs of the invention may be used to target cells and membranes, and to alter voltage-associated cell activities. In some embodiments, LAICs of the invention a UV LAIC of the invention may be used to sensitize cells to ultraviolet light. Such methods may be used to treat blindness and introduce visual perception to non-visible light.

In another aspect of the invention, a LAIC may be used to decrease the pH of a cell in which it is expressed. Such a technique may be used to treat alkalosis.

Another aspect of the invention includes methods of using light-activated proton pumps in conjunction with the use of LAICs of the invention for the coupled effect of hyperpolarization and intracellular alkalinization. For example, both phenomena can induce spontaneous spiking in neurons by triggering hyperpolarization-induced cation currents or pH-dependent hyper-excitability.

Another aspect of the invention includes a specific use of the Tetraselmis genus of chlorophyte. These are efficacious UV-activated ion channels and they express particularly well in mammalian membranes and perform robustly under mammalian physiological conditions. Another aspect of the invention is to generate sub-cellular voltage or pH gradients, particularly at synapses and in synaptic vesicles to alter synaptic transmission, and mitochondria to improve ATP synthesis.

In some embodiments, methods and LAICs of the invention may be used for the treatment of visual system disorders, for example to treat vision reduction or loss. A LAIC of the invention may be administered to a subject who has a vision reduction or loss and the expressed LAIC can function as light-sensitive cells in the visual system, thereby permitting a gain of visual function in the subject.

The present invention in some aspects, includes preparing nucleic acid sequences and polynucleotide sequences; expressing in cells and membranes polypeptides encoded by the prepared nucleic acid and polynucleotide sequences; illuminating the cells and/or membranes with suitable light, and demonstrating rapid depolarization of the cells and/or a change in conductance across the membrane in response to light, as well as rapid release from depolarization upon cessation of light. The ability to controllably alter voltage across membranes and cell depolarization with light has been demonstrated. The present invention enables light-control of cellular functions in vivo, ex vivo, and in vitro, and the light activated ion channels of the invention and their use, have broad-ranging applications for drug screening, treatments, and research applications, some of which are describe herein.

In illustrative implementations of this invention, the ability to optically perturb, modify, or control cellular function offers many advantages over physical manipulation mechanisms. These advantages comprise speed, non-invasiveness, and the ability to easily span vast spatial scales from the nanoscale to macroscale.

The reagents use in the present invention (and the class of molecules that they represent), allow, at least: currents activated by light wavelengths not useful in previous light-activated ion channels, light activated ion channels that when activated, permit effectively zero calcium conductance, and different spectra from older molecules (opening up multi-color control of cells).

### EXAMPLES

### Example 1

Studies were performed to prepare sequences and to express light-activated ion channels in cells, tissues, and subjects. Non-limiting exemplary methods are set forth Example 1. General methods also applicable to light-activated channel molecules and methods for their use are disclosed in publications such as US Published Application No. 2010/0234273, US Published Application No. 20110165681, Chow B Y, et. al. Methods Enzymol. 2011; 497:425-43; Chow, B Y, et al. Nature 2010 Jan. 7; 463(7277):98-102, the content of each of which is incorporated by reference herein.

Studies were performed to prepare sequences and to express light-activated ion channels in cells, tissues, and subjects. Non-limiting exemplary methods are set forth below.

### Plasmid Construction and Site Directed Mutagenesis.

Opsins were mammalian codon-optimized, and synthesized by Genscript (Genscript Corp., NJ). Opsins were fused in frame, without stop codons, ahead of GFP (using BamHI and AgeI) in a lentiviral vector containing the CaMKII promoter, enabling direct neuron transfection, HEK cell transfection (expression in HEK cells is enabled by a ubiquitous promoter upstream of the lentiviral cassette), and lentivirus production and transfection.

Amino acid sequences of various opsins were as follows: ChR66 (SEQ ID NO:2); ChR68 (SEQ ID NO:4); ChR77 (SEQ ID NO:6); ChR65 (SEQ ID NO:8), ChR2 (SEQ ID NO:10).

The ‘ss’ signal sequence from truncated MHC class I antigen corresponded to amino acid sequence (M)VPCTLLLLLAAALAPTQTRA (SEQ ID NO:11), DNA sequence gtcccgtgcacgctgctcctgctgttggcagccgccctggctccgactcagacgcgggcc (SEQ ID NO:12). The ‘ER2’ ER export sequence corresponded to amino acid sequence FCYENEV (SEQ ID NO:14), DNA sequence ttctgctacgagaatgaagtg (SEQ ID NO:13). The ‘KGC’ signal sequence corresponded to amino acid sequence KSRITSEGEYIPLDQIDINV (SEQ ID NO:16), DNA sequence of KGC signal sequence: aaatccagaattacttctgaaggggagtatatccctctggatcaaatagacatcaatgtt. (SEQ ID NO:15).

Halo point mutants for HEK cell testing were generated using the QuikChange kit [Stratagene, (Agilent Technologies, Santa Clara, Calif.)] on the Halo-GFP fusion gene inserted between BamHI and EcoRI sites in the pcDNA3.1 backbone [Invitrogen, (Life Technologies Corporation, Carlsbad, Calif.)]. All other point mutants for HEK cell testing were generated using the QuikChange kit [Stratagene, (Agilent Technologies, Santa Clara, Calif.)] on the opsin-GFP fusion gene inserted between BamHI and AgeI sites in a modified version of the pEGFP-N3 backbone [Invitrogen, (Life Technologies Corporation, Carlsbad, Calif.)]. All constructs were verified by sequencing.

### Neuron Culture, Transfection, Infection, and Imaging

All procedures involving animals were in accordance with the National Institutes of Health Guide for the care and use of laboratory animals and approved by the Massachusetts Institute of Technology Animal Care and Use Committee. Swiss Webster or C57 mice (Taconic, Hudson, N.Y. or The Jackson Laboratory, Bar Harbor, Mass.) were used. For hippocampal cultures, hippocampal regions of postnatal day 0 or day 1 mice were isolated and digested with trypsin (1 mg/ml) for ˜12 min, and then treated with Hanks solution supplemented with 10-20% fetal bovine serum and trypsin inhibitor (Sigma-Aldrich, St. Louis, Mo.). Tissue was then mechanically dissociated with Pasteur pipettes, and centrifuged at 1000 rpm at 4° C. for 10 min. Dissociated neurons were plated at a density of approximately four hippocampi per 20 glass coverslips, coated with Matrigel (BD Biosciences, Sparks, Md.). For cortical cultures, dissociated mouse cortical neurons (postnatal day 0 or 1) were prepared as previously described, and plated at a density of 100-200 k per glass coverslip coated with Matrigel (BD Biosciences, Sparks, Md.). Cultures were maintained in Neurobasal Medium supplemented with B27 [Invitrogen, (Life Technologies Corporation, Carlsbad, Calif.)] and glutamine. Hippocampal and cortical cultures were used interchangeably; no differences in reagent performance were noted.

Neurons were transfected at 3-5 days in vitro using calcium phosphate [Invitrogen, (Life Technologies Corporation, Carlsbad, Calif.)]. GFP fluorescence was used to identify successfully transfected neurons. Alternatively, neurons were infected with 0.1-3 μl of lentivirus or adeno-associated virus (AAV) per well at 3-5 days in vitro.

### HEK 293FT Cell Culture and Transfection

HEK 293FT cells [Invitrogen, (Life Technologies Corporation, Carlsbad, Calif.)] were maintained between 10-70% confluence in D10 medium (Cellgro, Manassas, Va.) supplemented with 10% fetal bovine serum [Invitrogen, (Life Technologies Corporation, Carlsbad, Calif.)], 1% penicillin/streptomycin (Cellgro, Manassas, Va.), and 1% sodium pyruvate (Biowhittaker, Walkersville, Md.)). For recording, cells were plated at 5-20% confluence on glass coverslips coated with Matrigel (BD Biosciences, Sparks, Md.). Adherent cells were transfected approximately 24 hours post-plating either with TransLT 293 lipofectamine transfection kits (Mirus, Madison, Wis.) or with calcium phosphate transfection kits [Invitrogen, (Life Technologies Corporation, Carlsbad, Calif.)], and recorded via whole-cell patch clamp between 36-72 hours post-transfection.

### Lentivirus Preparation

HEK293FT cells [Invitrogen, (Life Technologies Corporation, Carlsbad, Calif.)] were transfected with the lentiviral plasmid, the viral helper plasmid pΔ8.74, and the pseudotyping plasmid pMD2.G. The supernatant of transfected HEK cells containing virus was then collected 48 hours after transfection, purified, and then pelleted through ultracentrifugation. Lentivirus pellet was resuspended in phosphate buffered saline (PBS) and stored at −80° C. until further usage in vitro or in vivo. The estimated final titer is approximately 10⁹ infectious units/mL.

### In Vitro Whole Cell Patch Clamp Recording & Optical Stimulation

Whole cell patch clamp recordings were made using a Multiclamp 700B amplifier, a Digidata 1440 digitizer, and a PC running pClamp (Molecular Devices, Sunnyvale, Calif.). Neurons were bathed in room temperature Tyrode containing 125 mM NaCl, 2 mM KCl, 3 mM CaCl₂, 1 mM MgCl₂, 10 mM HEPES, 30 mM glucose, 0.01 mM NBQX and 0.01 mM GABAzine. The Tyrode pH was adjusted to 7.3 with NaOH and the osmolarity was adjusted to 300 mOsm with sucrose. HEK cells were bathed in a Tyrode bath solution identical to that for neurons, but lacking GABAzine and NBQX. Borosilicate glass pipettes (Warner Instruments, Hamden, Conn.) with an outer diameter of 1.2 mm and a wall thickness of 0.255 mm were pulled to a resistance of 3-9 MΩ with a P-97 Flaming/Brown micropipette puller (Sutter Instruments, Novato, Calif.) and filled with a solution containing 125 mM K-gluconate, 8 mM NaCl, 0.1 mM CaCl₂, 0.6 mM MgCl2, 1 mM EGTA, 10 mM HEPES, 4 mM Mg-ATP, and 0.4 mM Na-GTP. The pipette solution pH was adjusted to 7.3 with KOH and the osmolarity was adjusted to 298 mOsm with sucrose. Access resistance was 5-30 MΩ, monitored throughout the voltage-clamp recording. Resting membrane potential was ˜60 mV for neurons and ˜30 mV for HEK 293FT cells in current-clamp recording.

Photocurrents were measured with 500 ms light pulses in neurons voltage-clamped at −60 mV, and in HEK 293FT cells voltage-clamped at −30 mV. Light-induced membrane hyperpolarizations were measured with 500 ms light pulses in cells current-clamped at their resting membrane potential. Light pulses for all wavelengths except 660 nm and action spectrum characterization experiments were delivered with a DG-4 optical switch with 300 W xenon lamp (Sutter Instruments, Novato, Calif.), controlled via TTL pulses generated through a Digidata signal generator. Green light was delivered with a 575±25 nm bandpass filter (Chroma, Bellows Falls, Vt.) and a 575±7.5 nm bandpass filter (Chroma, Bellows Falls, Vt.). Action spectra were taken with a Till Photonics Polychrome V, 150 W Xenon lamp, 15 nm monochromator bandwidth.

Data was analyzed using Clampfit (Molecular Devices, Sunnyvale, Calif.) and MATLAB (Mathworks, Inc., Natick, Mass.).

### Example 2

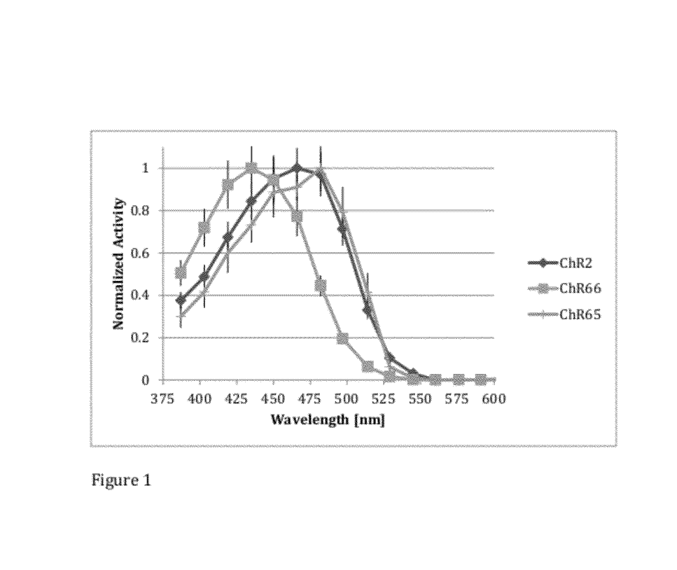

ChR2, ChR66 and Chr65 were expressed in HEK293 cells using methods described in Example 1. Normalized action spectrum were recorded in the cells under physiological conditions with the voltage clamped to −65 mV. Equal photon flux was sued at each wavelength. Results, which are shown in FIG. 1, demonstrate that the peak of normalized activity for the UV LAIC ChR66 was centered on shorter wavelengths than that recorded for ChR2 and ChR65.

### Example 3

Inward photocurrents elicited at peak wavelengths of light were tested and trafficking of LAIC sequences was examined using various signal and export sequences. Using methods set forth in Example 1, various signal and export sequences were included in vectors along with the LAIC sequences ChR66, ChR68, and ChR77, all of which are UV LAICs of the invention. ChR66-expressing cells were prepared using a vector that included the sequence of ChR66+KGC-ER2. ChR68 expressing cells were prepared using a vector that included the sequence of ChR68+KGC-ER2. Two sets of ChR77-expressing cells were prepared, one using a vector that included the sequence of ChR77+ER2, and another using a vector that included the sequence of ChR77+KGC-ER2.

Two sets of ChR2 expressing cells were also prepared, one with a vector that included the sequence of ChR2 and another that included the sequence of ChR2+KGC-ER2.

“ER2”, had a nucleic acid sequence set forth herein as SEQ ID NO:13 encoding the amino acid sequence set forth herein as SEQ ID NO:14.

“Kir2.1 (KGC-ER2), having the nucleic acid sequences set forth herein as SEQ ID NO:15 and SEQ ID NO:13 encoding the amino acid sequence set forth herein as SEQ ID NO:16 and SEQ ID NO:14.

The photocurrent was determined for each of the above LAIC constructs of the invention and for ChR2.

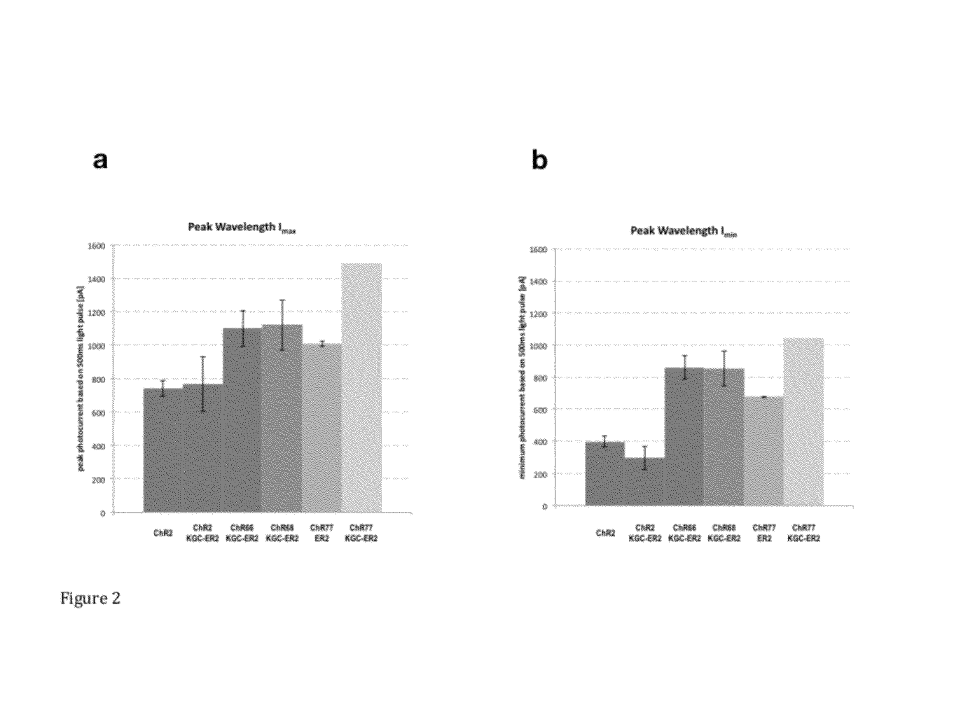

The inward photocurrents elicited at peak wavelength sensitivity of the channelrhodopsin in cultured hippocampal neurons were compared. 470±11 nm light was used for ChR2; 434±9 nm light was used for ChR66, ChR68, and ChR77. All illuminations consisted of 500 ms pulse of light at 10 mW mm⁻². KGC and ER2 are Kir2.1 trafficking sequences used to improve membrane expression of channelrhodopsin. The Iₘₐₓ (FIG. 2A) was defined as the maximum photocurrent during the 500 ms pulse interval. The Iₘᵢₙ (FIG. 2B) was defined as the minimum photocurrent at the end of the 500 ms pulse

### Results

The results indicated that UV-LAIC had higher or comparable peak photocurrent to ChR2 and 2-3 times higher stationary photocurrent than ChR2. This means UV-LAIC should be at least as efficacious as ChR2 in driving spikes and UV-LAIC higher steady state photocurrent indicated it was more reliable than ChR2 since there is very little channel “run-down”. Photocurrent results are set forth in FIG. 2.

### Example 4

Inward photocurrents were examined and trafficking of LAIC sequences by KGC-ER2 trafficking sequences was examined. Using methods set forth in Example 1, the KGC-ER2 signal and export sequences were included in vectors along with the LAIC sequences ChR66 and ChR68. ChR66-expressing cells were prepared using a vector that included the sequence of ChR66+KGC-ER2. ChR68 expressing cells were prepared using a vector that included the sequence of ChR68+KGC-ER2. ChR2 expressing cells were also prepared using a vector that included the sequence of ChR2+KGC-ER2.

KGC-ER2 had the nucleic acid sequences set forth herein as SEQ ID NO:15 and SEQ ID NO:13 encoding the amino acid sequence set forth herein as SEQ ID NO:16 and SEQ ID NO:14.

The photocurrent was determined for each of the above LAIC constructs of the invention and for ChR2.

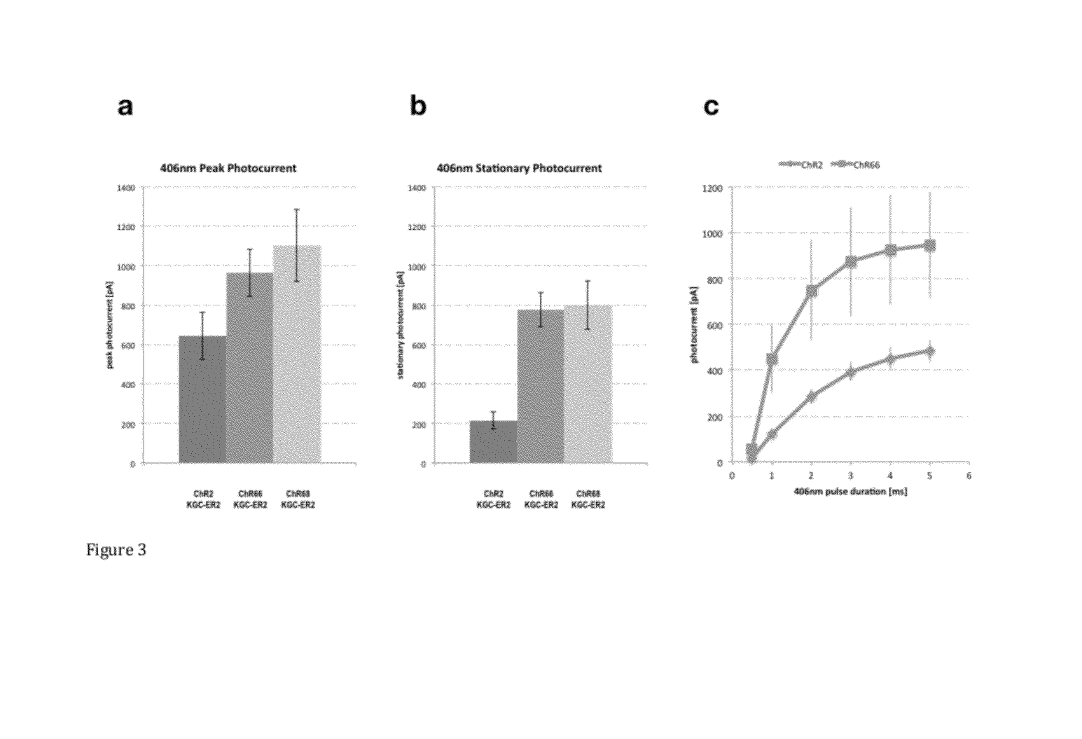

Results are shown in FIG. 3, which illustrates the comparison of inward photocurrent at 406±7 nm 10 mW mm⁻² in cultured hippocampal neurons. FIG. 3A shows the peak photocurrent response to 500 ms pulse. Showing ChR68 to have the highest photocurrent. FIG. 3B shows the minimum photocurrent at the end of 500 ms pulse. FIG. 3C shows results at 406 nm photon dose modulation with fixed power at 10 mW mm⁻² but varying pulse duration.

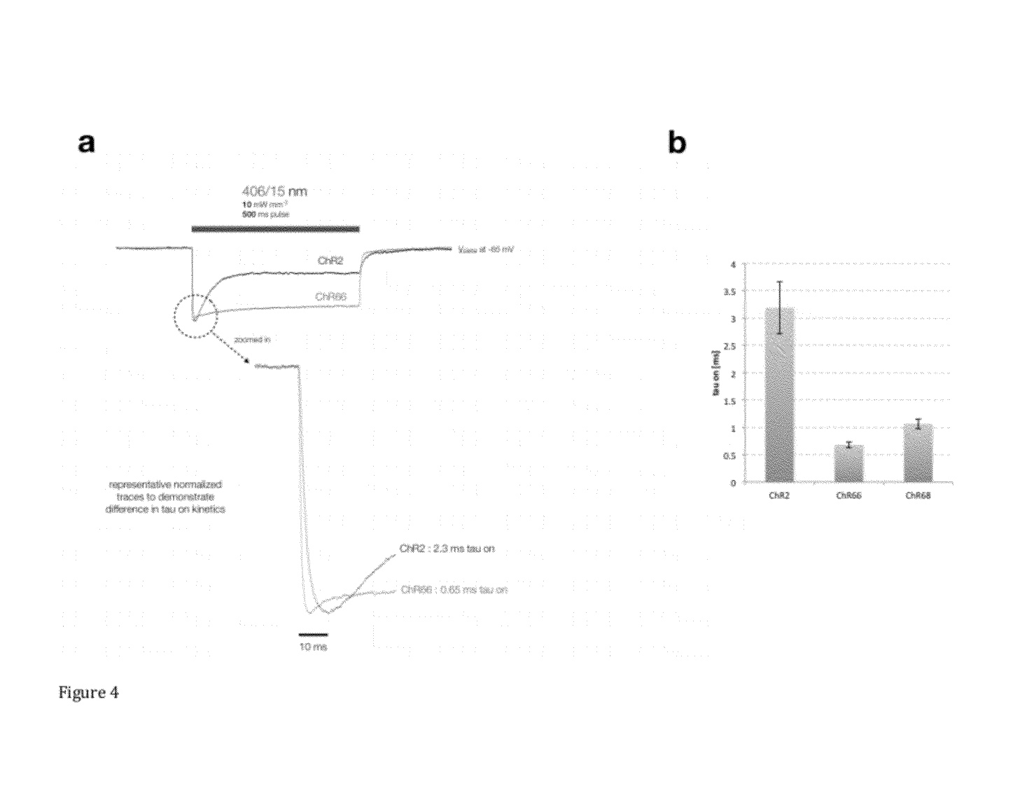

FIG. 4 also shows photocurrent onset kinetics at 406±7 nm 10 mW mm⁻² in cultured hippocampal neuron. KGC-ER2 trafficking sequence was used for all constructs.

FIG. 4A shows a representative photocurrent trace of ChR2 and ChR66 in voltage clamp mode. FIG. 4B provides a comparison of turn-on kinetics measured as single exponential fit of 10-90% initial transient photocurrent.

In general all channelrhodopsins have some sort of UV sensitivity (because the retinal chromophore present in all channelrhodopsin absorbs in the UV naturally. Ie. when retinal is not bound to opsin, but in free solution). FIGS. 3 and 4 demonstrate that UV LAIC has significantly more sensitivity in the UV and these can be distinguished from ChR2 functionally. FIG. 3 shows UV-LAIC had significantly higher photocurrent than ChR2 in the violet (406 nm). UV-LAIC can reliable drive spikes with brief pulses <2 ms at 406 nm whereas ChR2 cannot drive spikes. Thus, the results indicated that UV-LAIC have more photocurrent, and turned on much faster than ChR2 at 406 nm.

### Example 5

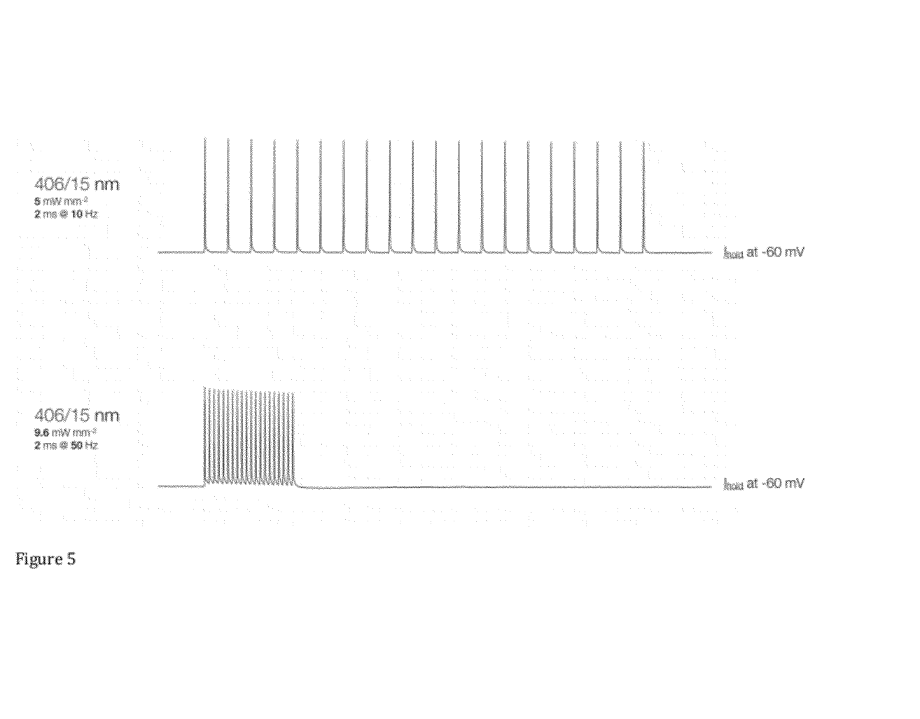

Constructs were made and ChR66-expressing cultured hippocampal neurons were prepared using methods set forth in Example 1. High fidelity spike trains were elicited in the cultured neurons at 406 nm. FIG. 5 shows the spike train. The results indicated that ChR66 has fast channel closing kinetics around 6.5 ms.

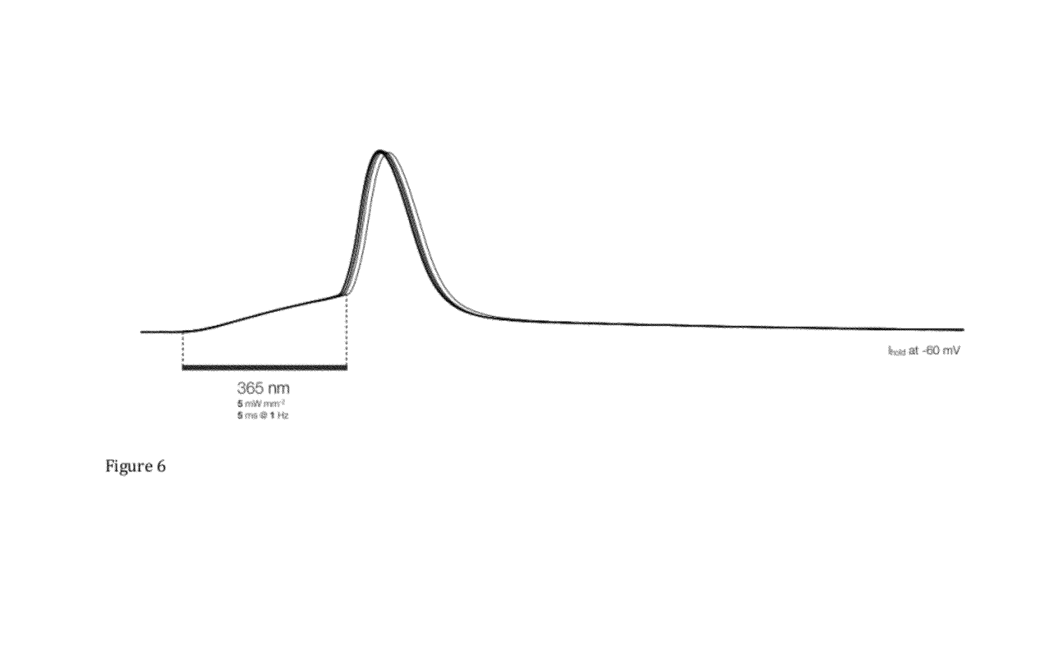

A spike train was driven in the CHR66-expressing cells using contact with UV light at 365 nm in a cultured hippocampal neuron. An exemplary spike train recording is provided in FIG. 6. The results indicated that ChR66 can drive high frequency spikes, at least 50 Hz (trace shown in FIG. 6), but it could be at any frequency up to 200 Hz. Not all channelrhodopsin can drive high frequency spikes, in fact most do not, ChR2 can only go up to 20 Hz reliably.

### Example 6

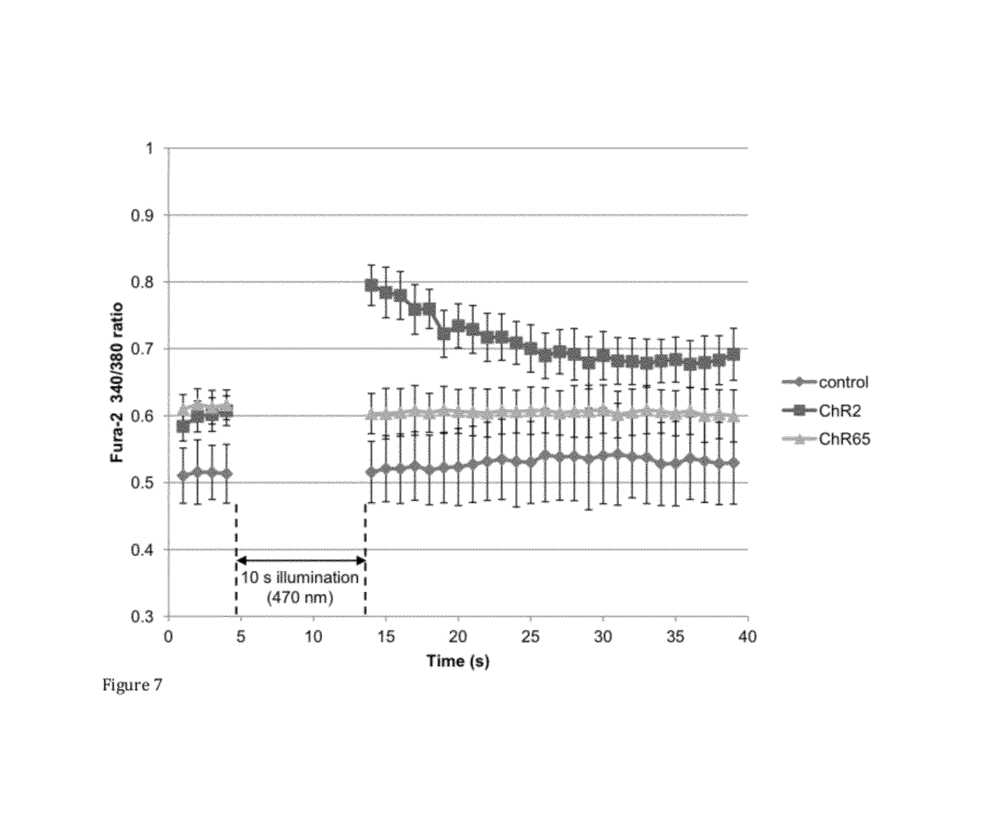

Constructs were made and Chr2-expressing HEK293 cells and ChR65-expressing HEK293 cells were prepared using methods set forth in Example 1. Fura2 calcium imaging was performed using standard procedures. See for example, Lin, J Y, et al., Biophys J. 2009 Mar. 4; 96(5):1803-14 and Kleinlogel, S. et al., Nat Neurosci. 2011 April; 14(4):513-8. Epub 2011 Mar. 13. Results of the imaging are shown in FIG. 7, provides results from ChR2 and ChR65 in HEK293, and results from a control test on untransfected cells. The extracellular medium was modified tyrode with 90 mM calcium. ChR65 did not exhibit any calcium permeability after 10 seconds of blue light (470 nm) illumination.

### Example 7

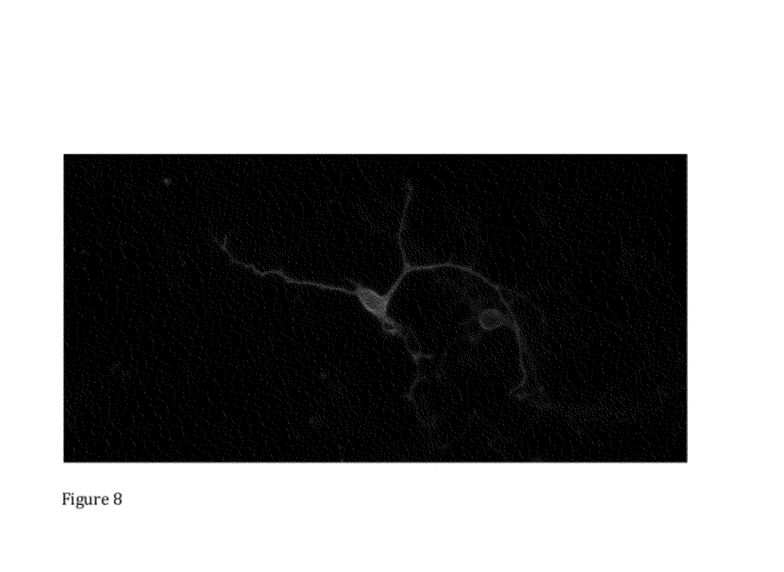

ChR66 was expressed in hippocampal cells. Two-three μg of plasmids and CaCl₂ were mixed to achieve final concentration of 250 mM calcium in 15 uL volume. Then 15 uL of 2×HBS was added to precipitate DNA-calcium phosphate. This mixture was used to transfect hippocampal neurons by incubating it for 20 minutes. After incubation period, several washing steps with plain media should be used to remove excess calcium phosphate precipitates. FIG. 8 shows a photomicrographic image of a hippocampal cell expressing ChR66 prepared as described. CaMKII is calcium/calmodulin-depending kinase II promoter is a neuron specific promoter commonly used, it is described in Dittgen, T, et al., PNAS Dec. 28, 2004 vol. 101 no. 52 18206-18211, epub Dec. 17, 2004.

### Example 8

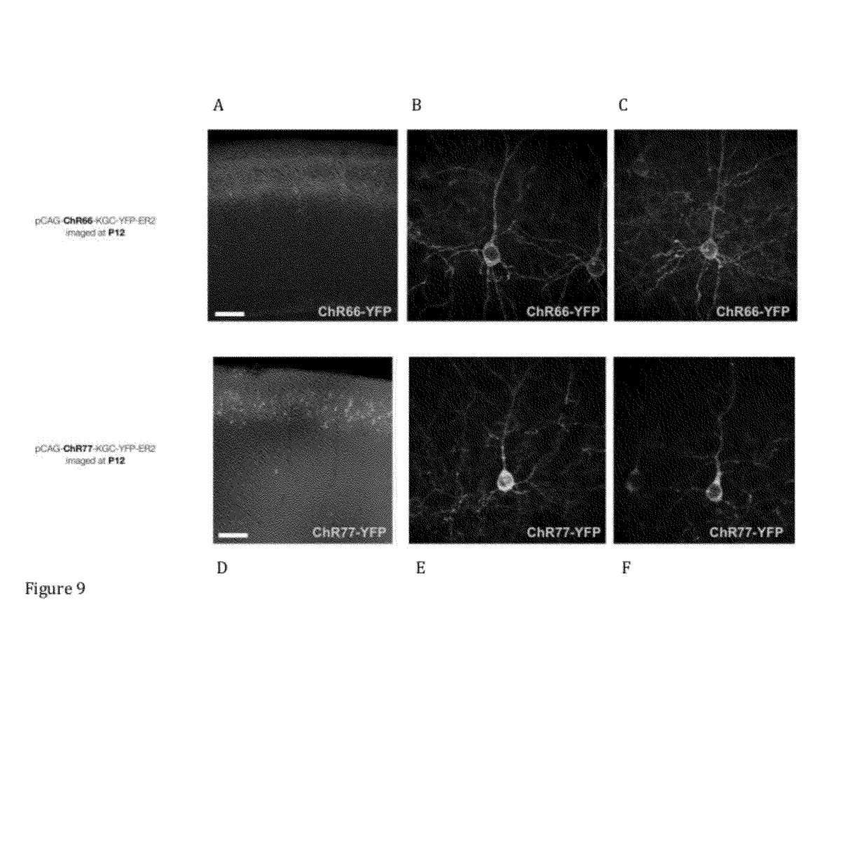

ChR66 and ChR77 were prepared and expressed in visual cortex. The constructs were: for ChR66 was pCAG-ChR66-KGC-YFP-ER2 and for ChR77 was pCAG-ChR66-KGC-YFP-ER2. Methods for preparation and expression, etc. included those set forth in Example 1. The KGC-ER2 trafficking sequence and sequence for GFP was included in both constructs. FIG. 9 is a photomicrographic image showing the resulting expression of ChR66 and ChR77 in visual cortex. FIGS. 9A-C show images of ChR66 expressed in visual cortex. FIG. 9A shows an image of the mouse visual cortex showing localization of the expressed ChR66 neurons via GFP fluorescence. FIGS. 9B and C show higher resolution images of the ChR66 expressed in individual cells of the visual cortex. FIG. 9D shows an image of the mouse visual cortex showing localization of the expressed ChR77 cells seen via GFP fluorescence. FIGS. 9E and F show a higher resolution image of the ChR77 expression in individual cells of the visual cortex. The results indicated delivery and expression of ChR66 and ChR77 in the visual cortex.

### Example 9

Genes described under (a), (b) and (c) were expressed in cells using methods provided below.

### Genes

The Tetraselmis striata gene referred to herein as ChR66 and having the amino acid sequence set forth herein as SEQ ID NO:2 and a mammalian codon-optimized DNA sequence set forth herein as SEQ ID NO:1;

b) The gene for Tetraselmis chuii referred to herein as ChR68, and having the amino acid sequence set forth herein as SEQ ID NO:4 and a mammalian codon-optimized DNA sequence set forth herein as SEQ ID NO:3; and

c) The gene for Brachiomonas submarina, referred to herein as ChR65 and having the amino acid sequence set forth herein as SEQ ID NO:8 and having a mammalian codon-optimized DNA sequence set forth herein as SEQ ID NO:7 are expressed in cells as follows.

### Methods

The opsin gene was cloned into a lentiviral or adeno-associated virus (AAV) packaging plasmid, or another desired expression plasmid, and then clone GFP downstream of the preferred gene, eliminating the stop codon of the opsin gene, thus creating a fusion protein.

The viral or expression plasmid contained either a strong general promoter, a cell-specific promoter, or a strong general promoter followed by one more logical elements (such as a lox-stop-lox sequence, which will be removed by Cre recombinase selectively expressed in cells in a transgenic animal, or in a second virus, thus enabling the strong general promoter to then drive the gene.

If using a viral plasmid, synthesize the viral vector using the viral plasmid.

If using a virus, as appropriate for gene therapy (over 600 people have been treated with AAV carrying various genetic payloads to date, in 48 separate clinical trials, without a single adverse event), inject the virus using a small needle or cannula into the area of interest, thus delivering the gene encoding the opsin fusion protein into the cells of interest. If using another expression vector, directly electroporate or inject that vector into the cell or organism (for acutely expressing the opsin, or making a cell line, or a transgenic mouse or other animal). Illuminate with light. For Tetraselmis, peak illumination wavelengths are 430 nm+50 nm. For Brachiomonas, peak illumination wavelengths are 470+/−50 nm.

To illuminate two different populations of cells (e.g., in a single tissue) with two different colors of light, first target one population with VChR1, and the other population with Tetraselmis, using two different viruses (e.g., with different coat proteins or promoters) or two different plasmids (e.g., with two different promoters). Then, after the molecule expresses, illuminate the tissue with 365±15 nm or 385±15 nm light to preferentially depolarize the Tetraselmis-expressing cells, and illuminate the tissue with 560±20 nm light, to preferentially depolarize the VChR1-expressing cells.

The above wavelengths illustrate typical modes of operation, but are not meant to constrain the protocols that can be used. Either narrower or broader wavelengths, or differently-centered illumination spectra, can be used. For prosthetic uses, the devices used to deliver light may be implanted. For drug screening, a xenon lamp or LED can be used to deliver the light.

Aspects of the invention include compositions of matter that have been reduced to practice, as described below:

Plasmids encoding for the above genes, have been prepared and used to deliver genes into cells, where the genes have been expressed. As an exemplary vector, lentiviruses carrying payloads encoding for the above genes have been prepared and used to deliver genes into cells resulting in expression of the LAIC in the cells. In addition, adeno-associated viruses carrying payloads encoding for the above genes have been prepared and used to deliver genes into cells, resulting in the expression of the LAIC in the cells. Cells have been prepared that express the LAIC genes set forth in Example 2. Animals have been prepared that include cells that express the LAIC genes set forth in Example 2.

It is to be understood that the methods, compositions, and apparatus which have been described above are merely illustrative applications of the principles of the invention. Numerous modifications may be made by those skilled in the art without departing from the scope of the invention.

Although the invention has been described in detail for the purpose of illustration, it is understood that such detail is solely for that purpose and variations can be made by those skilled in the art without departing from the spirit and scope of the invention which is defined by the following claims.

The contents of all references, patents and published patent applications cited throughout this application are incorporated herein by reference in their entirety.

We claim:

1. An isolated ultraviolet-light-activated ion channel polypeptide, wherein the ion channel when expressed in a membrane is activated by contact with ultraviolet light.

2. The isolated ultraviolet-light-activated ion channel polypeptide of claim 1, wherein the ultraviolet light has a wavelength of about 340 nm to about 400 nm.

3. A cell comprising the isolated ultraviolet-light-activated ion channel polypeptide of claim 1.

4-6. (canceled)

7. The cell of claim 3, further comprising one, two, three, four, or more additional light-activated ion channels, wherein at least one, two, three, four, or more of the additional light-activated ion channels is activated by contact with light that is not ultraviolet light and is not activated by light having a wavelength between about 340 and 400 nm.

8. A nucleic acid sequence that encodes the isolated ultraviolet-light-activated ion channel polypeptide of claim 1.

9. The nucleic acid sequence of claim 8, wherein the sequence comprises the sequence set forth as SEQ ID NO:1, SEQ ID NO:3, or SEQ ID NO:5.

10. The nucleic acid sequence of claim 8, wherein the nucleic acid sequence is a mammalian codon-optimized DNA sequence.

11. A vector comprising the nucleic acid sequence of claim 8.

12-14. (canceled)

15. A cell comprising the vector of claim 11.

16. The isolated ultraviolet-light-activated ion channel polypeptide of claim 1, wherein the ultraviolet-light-activated ion channel polypeptide sequence comprises conserved amino acids that correspond to G129, S133, T136, G141, and S169 of the amino acid sequence of ChR66, set forth herein as (SEQ ID NO:2).

17. The isolated ultraviolet-light-activated ion channel polypeptide of claim 1, wherein the ultraviolet-light-activated ion channel comprises the amino acid sequence of a wild-type or modified phytoplankton rhodopsin.

18. (canceled)

19. The isolated ultraviolet-light-activated ion channel polypeptide of claim 1, wherein the sequence of the light-activated ion channel polypeptide comprises an amino acid sequence set forth as SEQ ID NO:2, SEQ ID NO:4, or SEQ ID NO:6.

20-24. (canceled)

25. A method of depolarizing a cell, the method comprising,
contacting a cell comprising an isolated ultraviolet-light-activated ion channel with an ultraviolet light under conditions suitable to depolarize the cell and depolarizing the cell.

26. The method of claim 25, wherein the ultraviolet-light-activated ion channel activates in response to ultraviolet light in the range of about 340 nm to about 400 nm.

27. The method of claim 25, wherein the ultraviolet-light-activated ion channel polypeptide comprises conserved amino acids that correspond to G129, S133, T136, G141, and S169 of the amino acid sequence of ChR66, set forth herein as (SEQ ID NO:2).

28. The method of claim 25, wherein the ultraviolet-light-activated ion channel polypeptide sequence comprises an amino acid sequence of a wild-type or modified phytoplankton rhodopsin.

29-37. (canceled)

38. A method of assessing the effect of a candidate compound on a cell, the method comprising,
a) contacting a test cell comprising an isolated ultraviolet-light-activated ion channel with ultraviolet light under conditions suitable for depolarization of the cell;
b) contacting the test cell with a candidate compound; and
c) identifying the presence or absence of a change in depolarization or a change in a depolarization-mediated cell characteristic in the test cell contacted with the ultraviolet light and the candidate compound compared to depolarization or a depolarization-mediated cell characteristic, respectively, in a control cell contacted with the ultraviolet light and not contacted with the candidate compound;
wherein a change in depolarization or a depolarization-mediated cell characteristic in the test cell compared to the control indicates an effect of the candidate compound on the test cell.

39. The method of claim 38, wherein the ultraviolet light is in the range of about 340 nm to about 400 nm.

40-42. (canceled)

43. The method of claim 38, wherein the ultraviolet-light-activated ion channel polypeptide sequence comprises conserved amino acids that correspond to G129, S133, T136, G141, and S169 of the amino acid sequence of ChR66, set forth herein as (SEQ ID NO:2).

44. The method of claim 38, wherein the ultraviolet-light-activated ion channel polypeptide sequence comprises an amino acid sequence of a wild-type or modified phytoplankton rhodopsin.

45-134. (canceled)


SEQUENCE LISTING (16 sequences)

SEQ ID NO: 1 - DNA, 879 bp, Artificial Sequence
  Mammalian codon optimized sequence derived from Tetraselmis striata
atgttcgccatcaaccccgaatatatgaatgagactgtgctgctggacgaatgcaccccaatctacctggatattggaccactgtgggagcaggtggtggctagggtgactcagtggttcggagtgatcctgtctctggtgtttctgatctactacatctggaacacttacaaggctacctgtgggtgggaggaactgtacgtgtgcacagtggagttctgtaagatcatcatcgaactgtacttcgagtatactccccctgccatgatcttccagacaaatggacaagtgactccctggctgcggtatgctgagtggctgctgacatgccctgtgatcctgattcacctgtctaacattactgggctgaatgacgattacagtggccgcaccatgagcctgatcacatccgacctgggcggaatttgtatggctgtgaccgccgctctgagcaagggatggctgaaagccctgttctttgtgatcgggtgcggctacggagccagcaccttctacaacgccgcttgtatctacattgagtcctactataccatgccacagggaatttgcaggagactggtgctgtggatggccggggtgttctttacatcctggttcatgtttcctggcctgttcctggctggaccagaaggaacccaggccctgtcttgggctggaaccacaatcggacatacagtggccgacctgctgagtaagaacgcttggggaatgatcgggcactttctgagggtggagatccacaagcatatcatcatccatggagatgtgcggcgccccgtgacagtgaaggctctggggaggcaggtgagcgtgaattgtttcgtggacaaagaggaagaggaagaggatgagagaatc

SEQ ID NO: 2 - protein, 293 aa, Tetraselmis striata
MFAINPEYMNETVLLDECTPIYLDIGPLWEQVVARVTQWFGVILSLVFLIYYIWNTYKATCGWEELYVCTVEFCKIIIELYFEYTPPAMIFQTNGQVTPWLRYAEWLLTCPVILIHLSNITGLNDDYSGRTMSLITSDLGGICMAVTAALSKGWLKALFFVIGCGYGASTFYNAACIYIESYYTMPQGICRRLVLWMAGVFFTSWFMFPGLFLAGPEGTQALSWAGTTIGHTVADLLSKNAWGMIGHFLRVEIHKHIIIHGDVRRPVTVKALGRQVSVNCFVDKEEEEEDERI

SEQ ID NO: 3 - DNA, 885 bp, Artificial Sequence
  Mammalian codon optimized sequence derived from Tetraselmis chuii
atgggcttccagctgaaccccgaatatctgaatgagacaattctgctggacgattgcactccaatctacctgaacgtgggaccactgtgggagcagaaggtggctcggggaactcagtggttcggcgtgatcctgagcctggccttcctgatctactatatttggatcacatataaggctaccacatgtgggtgggaggaactgtacgtgtgcactattgagttctgtaaaattgtgatcgaactgtatttcgagttttccccccctgccatgatctaccagaccaatggagaagtgacaccctggctgagatatgctgagtggctgctgacctgccctgtgattctgatccacctgtctaacatcacaggcctgaatgacgattacagtggaagaactatgtctctgattaccagtgacctgggcggaatctgtatggctgtgaccagcgccctgtccaagggatggctgaaatggctgttctttgtgatcggctgctgttacggagccagcactttctatcacgccgctctgatctacatcgaatcctactataccatgccacatggagtgtgcaagaacatggtgctggccatggccgccgtgttcttcacctcttggttcatgtttcctgggctgtttctggctgggccagagggcacaaacgccctgagctgggctgggtccacaatcggccatactgtggccgatctgctgagtaagaatgcttggggaatgattgggcacttcctgagactggaaatccacaaacatatcattatccatggggacgtgaggagacccattactgtgaataccctgggccgggaggtgaccgtgtcttgttttgtggataaagaggaagaggacgaagatgagcgcatc

SEQ ID NO: 4 - protein, 295 aa, Tetraselmis chuii
MGFQLNPEYLNETILLDDCTPIYLNVGPLWEQKVARGTQWFGVILSLAFLIYYIWITYKATTCGWEELYVCTIEFCKIVIELYFEFSPPAMIYQTNGEVTPWLRYAEWLLTCPVILIHLSNITGLNDDYSGRTMSLITSDLGGICMAVTSALSKGWLKWLFFVIGCCYGASTFYHAALIYIESYYTMPHGVCKNMVLAMAAVFFTSWFMFPGLFLAGPEGTNALSWAGSTIGHTVADLLSKNAWGMIGHFLRLEIHKHIIIHGDVRRPITVNTLGREVTVSCFVDKEEEDEDERI

SEQ ID NO: 5 - DNA, 876 bp, Artificial Sequence
  Mammalian codon optimized sequence derived from Tetraselmis cordiformis
atgggttggaaaatcaatcctctgtactccgatgaggtcgcaatcctggaaatctgtaaagaaaatgaaatggtgtttgggccactgtgggagcagaagctggcacgggccctccagtggttcactgtcatcctgtcagctatttttctcgcatactacgtgtactccaccctgcgagcaacatgcggatgggaggaactctatgtctgtactgtggagttcaccaaagtggtcgtggaggtctacctggaatatgtgcccccttttatgatctaccagatgaacggccagcacactccttggctgcgatatatggagtggctgctcacctgcccagtgatcctgattcatctctccaacatcacagggctgaatgacgaatactctggtaggaccatgtctctgctcacaagtgatctgggcggaattgctttcgcagtcctgagcgccctcgctgtgggatggcagaagggtctgtacttcgggatcggttgcatctacggggccagcaccttctaccacgccgcttgtatctacatcgagtcataccatactatgcccgctggcaagtgcaaacggctggtcgtggcaatgtgcgccgtgttctttacctcttggttcatgtttccagccctgttcctcgctggccccgaatgttttgacggcctgacatggagcggcagcacaatcgcacacactgtcgctgatctgctcagcaaaaatatctggggcctgattggacactttctcagagtgggcatccacgagcatattctggtccatggagacgtgaggagacctatcgaagtgaccattttcggcaaggagaccagcctgaattgttttgtggagaatgatgatgaggaagatgatgtc

SEQ ID NO: 6 - protein, 292 aa, Tetraselmis cordiformis
MGWKINPLYSDEVAILEICKENEMVFGPLWEQKLARALQWFTVILSAIFLAYYVYSTLRATCGWEELYVCTVEFTKVVVEVYLEYVPPFMIYQMNGQHTPWLRYMEWLLTCPVILIHLSNITGLNDEYSGRTMSLLTSDLGGIAFAVLSALAVGWQKGLYFGIGCIYGASTFYHAACIYIESYHTMPAGKCKRLVVAMCAVFFTSWFMFPALFLAGPECFDGLTWSGSTIAHTVADLLSKNIWGLIGHFLRVGIHEHILVHGDVRRPIEVTIFGKETSLNCFVENDDEEDDV

SEQ ID NO: 7 - DNA, 981 bp, Artificial Sequence
  Mammalian codon optimized sequence derived from Brachiomonas submarina
atggaggcctacgcttatccagaactgctggggtctgccggcaggagtctgttcgctgctaccgtgcccgagaacatcagcgaatccacatgggtggacgccggctaccagcacttttggactcagagacagaatgagaccgtggtgtgcgaacactatacccatgccagctggctgatttcccatggcacaaaggctgagaaaactgccatgatcgcttgtcagtggttcgccttcggcagcgccgtgctgatcctgctgctgtacgcctggcacacctggaaggctacatccgggtgggaggaagtgtacgtgtgctgcgtggagctggtgaaagtgctgttcgagatctaccatgaaatccaccatccttgcaccctgtatctggtgacaggcaactttattctgtggctgcggtacggagagtggctgctgacttgtccagtgattctgatccacctgagcaatattactggactgaagaacgactacaacaagcgcaccatgcagctgctggtgtccgatatcggatgcgtggtgtggggagtgacagccgccctgtgctacgactataagaaatggattttcttttgcctgggcctggtgtacggatgtaacacctatttccacgccgctaaggtgtatatcgagggatatcatacagtgcccaagggggaatgcaggatcattgtgaaagtgatggccggcgtgttctactgttcttggacactgttccccctgctgtttctgctggggcctgagggcactggagccttttctgcttatggcagtactatcgcccacaccgtggctgatgtgctgtccaagcagctgtggggactgctggggcaccatctgcgggtgaaaattcacgagcatatcattatccacggaaatctgaccgtgtctaagaaagtgaaggtggccggggtggaggtggaaacacaggaaatggtggacagtactgaggaagatgctgtg

SEQ ID NO: 8 - protein, 327 aa, Brachiomonas submarina
MEAYAYPELLGSAGRSLFAATVPENISESTWVDAGYQHFWTQRQNETVVCEHYTHASWLISHGTKAEKTAMIACQWFAFGSAVLILLLYAWHTWKATSGWEEVYVCCVELVKVLFEIYHEIHHPCTLYLVTGNFILWLRYGEWLLTCPVILIHLSNITGLKNDYNKRTMQLLVSDIGCVVWGVTAALCYDYKKWIFFCLGLVYGCNTYFHAAKVYIEGYHTVPKGECRIIVKVMAGVFYCSWTLFPLLFLLGPEGTGAFSAYGSTIAHTVADVLSKQLWGLLGHHLRVKIHEHIIIHGNLTVSKKVKVAGVEVETQEMVDSTEEDAV

SEQ ID NO: 9 - DNA, 927 bp, Artificial Sequence
  Mammalian codon optimized sequence derived from Chlamydomonas reinhardtii
atggactatggcggcgctttgtctgccgtcggacgcgaacttttgttcgttactaatcctgtggtggtgaacgggtccgtcctggtccctgaggatcaatgttactgtgccggatggattgaatctcgcggcacgaacggcgctcagaccgcgtcaaatgtcctgcagtggcttgcagcaggattcagcattttgctgctgatgttctatgcctaccaaacctggaaatctacatgcggctgggaggagatctatgtgtgcgccattgaaatggttaaggtgattctcgagttcttttttgagtttaagaatccctctatgctctaccttgccacaggacaccgggtgcagtggctgcgctatgcagagtggctgctcacttgtcctgtcatccttatccacctgagcaacctcaccggcctgagcaacgactacagcaggagaaccatgggactccttgtctcagacatcgggactatcgtgtggggggctaccagcgccatggcaaccggctatgttaaagtcatcttcttttgtcttggattgtgctatggcgcgaacacattttttcacgccgccaaagcatatatcgagggttatcatactgtgccaaagggtcggtgccgccaggtcgtgaccggcatggcatggctgtttttcgtgagctggggtatgttcccaattctcttcattttggggcccgaaggttttggcgtcctgagcgtctatggctccaccgtaggtcacacgattattgatctgatgagtaaaaattgttgggggttgttgggacactacctgcgcgtcctgatccacgagcacatattgattcacggagatatccgcaaaaccaccaaactgaacatcggcggaacggagatcgaggtcgagactctcgtcgaagacgaagccgaggccggagccgtg

SEQ ID NO: 10 - protein, 309 aa, Chlamydomonas reinhardtii
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAV

SEQ ID NO: 11 - DNA, 60 bp, Mus musculus
gtcccgtgcacgctgctcctgctgttggcagccgccctggctccgactcagacgcgggcc

SEQ ID NO: 12 - protein, 21 aa, Mus musculus
MVPCTLLLLLAAALAPTQTRA

SEQ ID NO: 13 - DNA, 21 bp, Mus musculus
ttctgctacgagaatgaagtg

SEQ ID NO: 14 - protein, 7 aa, Mus musculus
FCYENEV

SEQ ID NO: 15 - DNA, 60 bp, Mus musculus
aaatccagaattacttctgaaggggagtatatccctctggatcaaatagacatcaatgtt

SEQ ID NO: 16 - protein, 20 aa, Mus musculus
KSRITSEGEYIPLDQIDINV